SEQUENCE LISTING (1 sequence)

SEQ ID NO: 1 - protein, 8 aa, Artificial Sequence
  cytosolic chicken ovalbumin (OVA) peptide
SIINFEKL

(19) United States
(12) Patent Application Publication
(10) Pub. No.: US 2022/0220197 A1
(43) Pub. Date: Jul. 14, 2022

(54) Cancer Treatment by Targeting Plexins in the Immune Compartment

(71) Applicants: VIB VZW, Gent (BE); KATHOLIEKE UNIVERSITEIT LEUVEN, K.U.LEUVEN R&D, Leuven (BE); ONCURIOUS NV, Leuven (BE)

(72) Inventors: Massimiliano Mazzone, Kessel-Lo (BE); Ana Oliveira, Leuven (BE); Ward Celus, Kinrooi (BE); Rosa Martín-Pérez, Hevelee (BE); Catelijne Stortelers, Gent (BE)

(21) Appl. No.: 17/614,430

(22) PCT Filed: May 28, 2020
(86) PCT No.: PCT/EP2020/064905

§ 371 Date: Nov. 26, 2021

(30) Foreign Application Priority Data
May 28, 2019 (EP) 19176944.7

(51) Int. Cl. C07K 16/28; A61K 39/395; A61P 35/00

(52) U.S. Cl. CPC C07K 16/28; C07K 16/2815; C07K 16/2851; C07K 2317/31; A61P 35/00; C07K 2317/76; C07K 2317/569; A61K 39/3955

(57) ABSTRACT

The invention relates to compounds inhibiting plexin-A2 and/or plexin-A4, with said compounds specifically targeting plexin-A2 and/or plexin-A4 on or in CD8-positive (CD8+) T-cells. Medical uses of such compounds are also part of the invention.

### CROSS REFERENCE TO RELATED APPLICATIONS

This application is a national phase entry under 35 U.S.C. § 371 of International Patent Application PCT/EP2020/064905, filed May 28, 2020, designating the United States of America and published in English as International Patent Publication WO 2020/239945 on Dec. 3, 2020, which claims the benefit under Article 8 of the Patent Cooperation Treaty to European Patent Application Serial No. 19176944.7, filed May 28, 2019, the entireties of which are hereby incorporated by reference.

### FIELD OF THE INVENTION

The invention relates to compounds inhibiting plexin-A2 and/or plexin-A4, with said compounds specifically targeting plexin-A2 and/or plexin-A4 on or in CD8-positive (CD8+) T-cells. Medical uses of such compounds are also part of the invention.

### BACKGROUND OF THE INVENTION

After years of evidence that even the best cytotoxic regimens were unsuccessful in curing late stage malignancies, immunotherapy emerged as a promising treatment for cancer patients (for a review, see e.g. Decker et al. 2017, Front Immunol 8:829). These offer a rapid and robust activity (e.g., anti-PD-1 treatment in melanoma (Hamid et al. 2013, N Engl J Med 369:134-144), mostly because, once the immune system is activated, it can potentiate a self-propagating and adaptable response (Chen & Mellman 2013, Immunity 39:1-10). Indeed, immunotherapy leads to durable clinical responses, but only in a fraction of patients and tumor types (Sharma & Allison 2015, Science 348:56-61). The so-called immunologically “cold” tumors are characterized by an enrichment in immunosuppressive cytokines, high number of regulatory T (Treg) cells, and few T helper 1 (T_{H}1), natural killer (NK), cytotoxic (CD8+) T lymphocytes (CD8+ T-cells or CTLs) and antigen-presenting cells (APCs) (Nagarsheth et al. 2017, Nat Rev Immunol 17:559-572). In these tumors, the immunosuppressive tumor microenvironment (TME) can hamper the efficiency of immunotherapy independently from their antigenicity (Joyce & Fearon 2015, Science 348:74-80; Hugo et al. 2016, Cell 165:35-44; Spranger et al. 2016, PNAS 113:E7759-E7768). Thus, a deeper understanding of the mechanisms defining the immune landscape of a tumor could attain a broader and more durable response to this therapeutic option.

Plexins are large transmembrane glycoproteins that function as the receptors/ligands for the axon guidance proteins named semaphorins (Perala et al. 2012, Differentiation 83:77-91; Battistini & Tamagnone 2016, Cell Mol Life Sci 73:1609-1622. Accordingly, for several years, the research on this topic was focused on the nervous system, where they play a bifunctional role, having the capacity to exert both repulsive and attractive effects (He et al. 2002, Sci STKE 2002(119):re1). These chemoattractant properties together with the discovery of their role in immune responses in both physiological and pathological conditions (Kumanogoh & Kikutani 2013, Nat Rev Immunol 13:802-814; Roney et al. 2013, Protein Cell 4:17-26) led to the study of semaphorin signaling in the TME (Capparuccia et al. 2009, J Cell Sci 122:1723-1736). It was already demonstrated that blocking of Sema3A signaling plays a key role in restoring anti-tumor immunity by impeding tumor associated macrophages (TAMs) to enter hypoxic niches (Casazza et al. 2013, Cancer Cell 24:695-709). Additionally, it was shown that Sema4A signaling promotes Treg cell stability in the TME (Delgoffe et al. 2013, Nature 501:252-256). Plexin A4 (PlxnA4) is a member of class A plexins (Fujisawa 2004, J Neurobiol 59:24-33) that acts as the interactor of class 6 semaphorins (Battistini et al. 2013, Cell Mol Life Sci 73:1609-1622). Together with neuropilin 1 (Nrp1), it can also function as a co-receptor for class 3 semaphorins (Fujisawa et al. 2004, J Neurobiol 59:24-33). In the central nervous system, PlxnA4 was found to be a potent mediator of axon-repulsive activities by the direct binding to class 6 transmembrane semaphorins, Sema6A and Sema6B (Suto et al. 2005, J Neurosci 25:3628-3637; Tawarayama et al. 2010, J Neurosci 30:7049-7060). Nevertheless, in the immune system, it has different functions. On one hand, PlxnA4 seems to have a positive role in Toll-like receptor (TLR)-mediated signaling and macrophage cytokine production, as Plxna4-deficient mice have attenuated TLR-mediated inflammation, including septic shock (Wen et al. 2010, J Exp Med 207:2943-2957). On the other hand, the same mice showed enhanced T cell priming and exacerbated disease in a mouse model of experimental autoimmune encephalomyelitis (EAE) (Yamamoto et al. 2008, Int Immunol 20:413-420).

International Patent Publications WO 2001/014420, WO 2012/114339 and WO 2015/037009 are related to Plexin-A4. WO 2001/014420 discloses plexin-A4 as novel member of the plexin family; WO 2012/114339 focuses on molecules binding to type A plexins and inhibiting proliferative signals trough the type A plexin receptor without interfering with binding of the type A plexin to neuropilin or semaphorin 6A. WO 2015/037009 discloses antibodies binding to Plexin-A4.

The role of Plexin A2 (PlxnA2) in the immune system is unknown. The ligands of PlxnA2 appear to overlap with those of PlxnA4, and PlxnA2 was described as a repulsive guidance molecule in the central nervous system (Suto et al. 2007, Neuron 53:535-547; Shim et al. 2012, Mol Cell Neurosci 50:193-200).

### SUMMARY OF THE INVENTION

The invention in one aspect relates to compounds capable of inhibiting plexin-A2 and/or plexin-A4, wherein said compounds are specifically targeting plexin-A2 and/or plexin-A4 on or in CD8-positive (CD8+) T-cells. Such compound inhibiting plexin-A2 and/or plexin-A4 include compounds comprising a polypeptide, a polypeptidic agent, or an aptamer binding to plexin-A2 and/or plexin-A4; compounds which are inducing degradation of plexin-A2 and/or of plexin-A4; or compounds which are interfering with expression of plexin-A2 and/or of plexin-A4. In particular, such compounds are selected from a polypeptide comprising an immunoglobulin variable domain, an antibody or a fragment thereof, an alpha-body, a nanobody, an intrabody, an aptamer, a DARPin, an affibody, an affitin, an anticalin, a monobody, a bicyclic peptide, a PROTAC, or a LYTAC; or is chosen from any combination of any of the foregoing; wherein the compounds are binding plexin-A2 and/or plexin-A4. Any of these compounds may further comprise a moiety binding to a CD8+ T-cell-specific surface marker different from plexin-A2 and/or plexin-A4; in particular the CD8+ T-cell-specific surface marker different from plexin-A2 and/or plexin-A4 is CD8 or CD69.

In one embodiment, any of the above compounds is specifically targeting plexin-A2 and/or plexin-A4 on or in CD8+ T-cells in a tumor and/or in the tumor micro-environment of a subject having a tumor. The specificity for a tumor and/or the tumor-environment can in one embodiment be achieved by means of intra- or peri-tumoral administration of the compounds of the invention. Alternatively, the compounds of the invention can be delivered by means of a carrier, wherein the cargo of the carrier is the compound inhibiting plexin-A2 and/or plexin-A4, wherein the carrier is targeting its cargo to the tumor and/or tumor micro-environment, and/or wherein release of the cargo from the carrier can be controlled to occur in the tumor and/or in the tumor micro-environment. Such carriers include for instance viruses, oncolytic viruses, cells adoptively transferred to the subject, or exosomes, nanoparticles or microbubbles.

In another aspect of the invention, the present invention provides compounds that (a) inhibit plexin-A2 and/or plexin-A4, and (b) bind to a CD8-positive (CD8+) T-cell. In a further embodiment, the compound binds to a surface marker of a CD8-positive T-cell. In a further particular embodiment, the present invention provides a compound that (a) inhibits plexin-A2 and/or plexin-A4, particularly plexin-A4, and (b) binds to CD8.

A further aspect of the invention relates to pharmaceutical compositions comprising any of the above compounds inhibiting plexin-A2 and/or plexin-A4 according to the invention. Such compositions may optionally comprise an anticancer agent.

The invention also envisages any of the compounds inhibiting plexin-A2 and/or plexin-A4 according to the invention, or any of the pharmaceutical compositions according to the invention to be suitable for use as medicament; for use in treating, inhibiting, or suppressing a tumor or cancer; or for use in treating, inhibiting, or suppressing a tumor or cancer, further in combination with surgery, radiation, chemotherapy, targeted therapy, immunotherapy, or a further anticancer agent.

In a further aspect, the invention relates to pharmaceutical kits comprising as one component at least one of the compounds inhibiting plexin-A2 and/or plexin-A4 according to the invention, or at least one of the pharmaceutical compositions according to the invention.

### DESCRIPTION OF THE DRAWINGS

FIGS. 1A-1C. Hypoxic upregulation of Sema6B. Top panel (FIG. 1A): Expression of Sema 6B in tumor-associated macrophages (TAMs) from subcutaneous LLC tumors under hypoxic and normoxic conditions. Middle (FIG. 1B) and bottom (FIG. 1C) panel: Sema6B mRNA expression in distinct tumor cell lines (LLC, E0771 and Panc05 in middle panel; GL261, KR158B and CT2A in bottom panel) grown under hypoxic and normoxic conditions. Expression is normalized to HPRT house-keeping gene. ***p<0.001 versus WT. All graphs show mean±SEM.

FIGS. 2A-2M. Loss of PlxnA4 in the stroma abates tumor growth without affecting TAMs phenotype and tumor vasculature. (FIGS. 2A-2B) Subcutaneous LLC tumor growth (FIG. 2A) and weight (FIG. 2B) in mice with a full deletion of Plxna4 (KO in short) and control littermates (WT in short); (FIGS. 2C-2D) Subcutaneous B16-F10 tumor growth (FIG. 2C) and weight (FIG. 2D) in mice Plxna4 KO and control littermates; (FIG. 2E) F4/80 quantification showing TAMs infiltration of end-stage subcutaneous LLC tumors in WT and KO Plxna4 mice; (FIG. 2F) Expression of M1 (11-12, Cxcl10, Tnfa, Cd80) and M2 (Cc117, 11-10, Mrc1, Cxc112) markers in TAMs sorted from subcutaneous LLC tumors growing in WT and KO Plxna4 mice; (FIGS. 2G-2I) Histological analysis (FIGS. 2G-2H) and micrographs (FIG. 2I) of LLC tumor sections stained for F4/80 and pimonidazole (PIMO), showing tumor hypoxia (FIG. 2G) and TAMs infiltration of hypoxic tumor regions (FIG. 2H) in WT and KO Plxna4 mice. (FIGS. 2J-2M) Histological quantifications of tumor vessels on thin sections of LLC tumors growing in WT and Plxna4 KO mice showing vessel density (FIG. 2J and FIG. 2L), percentage of lectin-FITC⁺ perfused vessels over total number of CD34⁺ vessels (FIG. 2K), and percentage of NG2⁺ pericyte-covered vessels over the total number of CD31⁺ vessels (FIG. 2M). n=4-5 (FIGS. 2A, 2C-2E, 2G-2M) and n=8 (FIG. 2B, two independent experiments pooled). **p<0.01, and ****p<0.0001 versus WT. ns, not-significant versus WT. Scale bars: 100 μm. All graphs show mean±SEM.

FIGS. 3A-3H. Deletion of PlxnA4 in the immune system reduces tumor growth in orthotopic models and increases CD8+ T-cell infiltration. (FIGS. 3A-3B) Orthotopic E0771 breast cancer model tumor growth (FIG. 3A) and weight (FIG. 3B) in lethally irradiated WT mice reconstituted with WT (WT→WT) or Plxna4 KO (KO→WT) bone marrow cells; (FIG. 3C) FACS analysis of CD8⁺ and CD4+ T cell subsets infiltrating E0771 tumors 17 days after injection in WT→WT and Plxna4 KO→WT mice (n=4-6) CD4+ T cell subsets: total T helper, regulatory T (expressing FoxP3), Th2 (expressing GATA3) and Th1 (expressing Tbet) cells. n=5-6. Dashed lines represent FMO controls. MFI, Median Fluorescent Intensity; FMO, Fluorescence Minus One. (FIGS. 3D-3E) Orthotopic GL261 glioma model tumor volume (FIG. 3D) 23 days after stereotactic injection and BLI-assessed relative tumor size (FIG. 3E) at day 15 after injection in WT→WT and Plxna4 KO→WT mice; (FIG. 3F) Quantification of CD8+-stained GL261 tumor sections from WT→WT and KO→WT mice (n=7-11). (FIG. 3G) LLC model tumor growth and weight (FIG. 3H) in lethally irradiated WT mice reconstituted with WT (WT→WT) or Plxna4 KO (KO→WT) bone marrow cells. *p<0.05 and **p<0.01 versus WT→WT. Scale bars: 50 jam. All graphs show mean±SEM.

FIGS. 4A-4L. PIxnA4 loss in CD8+ T-cells increases their migratory capacity. (FIG. 4A) Plxna4 expression in CD8+ T-cells sorted from subcutaneous LLC tumor-bearing WT mice. (FIG. 4B) Plxna4 expression in sorted CD8+ T-cells before and after ex-vivo activation with CD3/CD28 dynabeads for 4 days; (FIG. 4C) Migration of WT and Plxna4-KO CD8+ T-cells towards CCL21 and CCL19; (FIGS. 4D-4F) Homing of WT and Plxna4 KO CD8+ T-cells to the lymph nodes assessed by FACS (FIG. 4D), quantification by histology (FIG. 4E) and a representative micrograph (FIG. 4F); (FIGS. 4G-4H) FACS analysis of CD8+ T-cells in the draining LNs of WT and Plxna4 KO mice bearing subcutaneous LLC tumors (FIG. 4G), or in chimeric WT→WT and Plxna4 KO→WT mice bearing orthotopic E0771 tumors (FIG. 4H); (FIG. 4I) Migration of WT and Plxna4-KO CD8+ T-cells towards CXCL9 and CXCL10. For the in vivo experiments, n=4 (FIG. 4A) and n=5-6 (FIGS. 4D-4H). (FIG. 4J) Homing of naïve WT and Plxna4 KO CD8⁺ T cells to the lymph nodes of WT mice treated with vehicle or FTY720 (fingolimod). (FIGS. 4K-4L) Tumor homing of activated WT and Plxna4 KO OT-I T cells to LLC-OVA tumor-bearing mice (FIG. 4J) or B16-F10-OVA tumor-bearing mice (FIG. 4K) assessed by flow cytometry 24 hours (FIGS. 4J-4K) and 48 hours (FIG. 4K) after T cell injection. In vitro results (FIGS. 4B-4C, and FIG. 4I) are representative of at least two independent experiments. *p<0.05, **p<0.01, ***p<0.001 and ****p<0.0001 versus LNs (FIG. 4A), naïve CD8+ T-cells (FIG. 4B), WT (FIGS. 4C-4G), WT→WT control (FIG. 4H), or WT OT-I T-cells (FIGS. 4K, and 4L). Scale bar: 100 μm. All graphs show mean±SEM.

FIGS. 5A-5G. PlxnA4 KO CD8+ T-cells have increased proliferation index and present more effective anti-tumor responses. (FIGS. 5A-5C) Ex-vivo proliferation of WT and Plxna4 KO Violet Cell Tracer-labelled splenocytes upon CD3/CD28 activation showing percentage of CD8+ T-cells (FIG. 5A), proliferation index (FIG. 5B) and a representative histogram of Violet Cell Tracer fluorescence intensity, gated on CD8⁺ cells, after 4 days in culture (FIG. 5C). (FIG. 5D) Cytotoxicity marker FACS analysis of splenocytes derived CD8+ T-cells upon CD3/CD28 activation showing IFNγ and GrzmB expression in WT and Plxna4 KO CD8⁺ T cells, after 4 days in culture. Representative of at least two independent experiments. (FIGS. 5E-5F) FACS analysis of CD69 activation marker in the draining LNs of WT and Plxna4 KO subcutaneous LLC tumor-bearing mice (FIG. 5E) or in chimeric WT→WT and Plxna4 KO→WT mice bearing orthotopic E0771 tumors (FIG. 5F); For the in vivo experiments, n=5-6 (FIGS. 5D-5F). In vitro results (FIGS. 5A-5C) are representative of at least two independent experiments. *p<0.05, **p<0.01 and ****p<0.0001 versus WT (FIGS. 5A-5D) or WT→WT control (FIG. 5E). #p<0.0001 versus PBS control (FIG. 5F). (FIG. 5G) FACS analysis of B16-F10-OVA tumors 24 hours after intratumoral injection of WT and Plxna4 KO OT-I T cells. All graphs show mean±SEM. Dashed lines represent FMO controls. MFI, Median Fluorescent Intensity; FMO, Fluorescence Minus One; ns, not-significant versus WT control.

FIGS. 6A-6F. Adoptive T cell transfer (ACT) of WT and KO OT-1 CD8⁺ T cells in LLC-OVA or B16-F10-OVA tumor bearing mice. (FIG. 6A) Tumor growth model in subcutaneous LLC-OVA tumor bearing mice, with ACT at day 5 (FIG. 6A) after tumor inoculation. (FIG. 6B) Tumor growth model in subcutaneous B16-F10-OVA tumor bearing mice, with ACT at day 13 (FIG. 6B) after tumor inoculation. (FIGS. 6A-6B) Comparison of WT and KO OT-I CD8+ T cells and PBS as control. (FIG. 6C) Survival effect (Kaplan-Meier overall survival curves) of ATC with WT and KO OT-I CD8⁺ T cells in subcutaneous B16-F10-OVA tumor bearing mice.

(FIG. 6D) weight and (FIG. 6E) representative images of end-stage B16-F10-OVA tumor tumors; (FIG. 6E):scale bar=2 cm. (FIG. 6F) FACS analysis of intratumoral OT-I T cells in B16-F10-OVA tumors isolated 4 days after ACT.

Groups n=5-6. #p<0.0001 versus PBS control, **p<0.01. All graphs show mean±SEM.

FIGS. 7A-7H. PlexinA2-specific deletion in CD8+ T cells increases anti-tumor immunity. (FIGS. 7A-7B) PlexinA2 mRNA expression in CD8+ T-cells in tissues of normal and tumor-bearing mice. (FIG. 7A) PlexinA2 mRNA expression is high in FACS sorted CD8+ T cells from blood as compared to LNs and spleen of healthy WT mice. (FIG. 7B) PlexinA2 is highly expressed in FACS sorted CD8+ T cells from blood while expressed at a lower level in sorted CD8+ T cells from lymph node (LN), tumor-draining LNs, spleen and primary tumor of subcutaneous LLC tumor-bearing WT mice. (FIGS. 7C-7D) Effect of CD8-positive T-cell-specific deletion of PlxnA2 on tumor volume (FIG. 7C) and tumor weight (FIG. 7D) in a subcutaneous MC38 colon adenocarcinoma tumor model. (FIGS. 7E-7F) Effect of CD8-positive T-cell-specific deletion of PlxnA2 on tumor volume (FIG. 7E) and tumor weight (FIG. 7F) in a orthotopic E0771 breast tumor model (FIGS. 7G-7H) Tumor-infiltration of CD8+ T cells in PIxA2 lox/lox and PlxnA2+/+ mice containing E0771 tumors (percentage of live cells). FACS analysis of E0771 tumors (sacrifice at day 16) with a specific deletion of CD8+ T cells showed increased number of blood circulating CD8+ T-cells (FIG. 7H) and more CD8+ T-cell infiltration in the primary tumor (FIG. 7G) as compared to their littermate controls.

FIGS. 8A-8D. PlxnA4 expression is dynamically regulated in CD8⁺ T lymphocytes. (FIGS. 8A-8C) Plxna4 expression in CD8+ T cells sorted from different tissues in LLC tumor-bearing WT mice (FIG. 8A), in circulating CD8+ T cells sorted from healthy, orthotopic B16-F10 and subcutaneous LLC tumor-bearing WT mice (FIG. 8B), and in sorted CD8+CD44⁻ and CD8⁺CD44+ cells from the circulation of B16-F10 tumor-bearing WT mice (FIG. 8C). (FIG. 8D) Plxna4 expression in purified CD8⁺ WT T cells before and after in vitro activation with CD3/CD28 beads. For the in vivo experiments, n=3-4 mice per group were used (FIGS. 8A-8C). In vitro results (FIG. 8D) were performed in triplicates and are representative of two independent experiments. *p<0.05, **p<0.01 and ***p<0.001 versus circulating CD8⁺ T cells (FIG. 8A), circulating CD8⁺ T cells in healthy mice (FIG. 8B), circulating CD8⁺CD44⁻ cells (FIG. 8C) and naïve CD8⁺ T cells (FIG. 8D). All graphs show mean±SEM.

FIGS. 9A-9B. Plxna4 expression is upregulated in circulating CD8⁺ T cells of melanoma patients. (FIG. 9A and FIG. 9B) Expression of Plxna4 in isolated CD8+ T cells from the circulation of treatment-naïve melanoma patients and healthy controls (FIG. 9A) and from naïve and ICIs-treated melanoma patients (FIG. 9B). n=6 healthy controls, n=23 naïve melanoma patients and n=14 αPD-1/CTLA-4-treated melanoma patients. *p<0.05 versus circulating CD8+ T cells in healthy individuals (FIG. 9A) and in naïve melanoma patients (FIG. 9B). All graphs show mean±SEM.

FIGS. 10A-10B. (FIG. 10A) Binding of bispecific VHHs and control VHHs to HEK293 cells recombinantly expressing human Plexin-A4. Details of the VHHs are listed in Table 4 herein. (FIG. 10B) Western blot confirmation of recombinant expression of human Plexin-A4 in HEK293 cells. Lane 1: molecular weight marker; lane 2: lysate of HEK293 cells recombinantly expressing human Plexin-A4; lane 3: lysate of HEK293 cells transfected with empty vector (not expressing human Plexin-A4). Plexin-A4 was detected by using R&D Systems antibody MAB58561.

FIGS. 11A-11B. (FIG. 11A) Binding of bispecific VHHs and control VHHs to CD4+ T-cells. (FIG. 11B) Binding of bispecific VHHs and control VHHs to CD8+ T-cells. Details of the VHHs are listed in Table 4 herein.

FIG. 12. Competition for binding human Plexin-A4 between human Sem6A and the indicated VHHs. Details of the VHHs are listed in Table 4 herein.

FIG. 13. Simultaneous binding of the indicated VHHs to human Plexin-A4 and CD8 as determined using biolayer interferometry. Details of the VHHs are listed in Table 4 herein.

### DETAILED DESCRIPTION OF THE INVENTION

Escaping from the immune system is a hallmark of cancer as immune cells carry the potential to limit tumor progression. Nevertheless, the immunosuppressive tumor microenvironment can hamper the efficiency of immunotherapy. In work leading to the present invention, the potential of targeting of plexin-A4 (PlxnA4) and plexin-A2 (PlxnA2), both known as repulsive guidance molecules, to increase anti-tumor immunity was shown. PlxnA4 or PlxnA2 deficiency in the stroma reduced tumor growth in several tumor models and this reduction was accompanied by an increased infiltration of CD8-positive (CD8+) T-cells. Furthermore, deletion of PlxnA4 or PlxnA2 in CD8+ T-cells was shown to be sufficient to increase their migratory capacity towards the lymph nodes/tumors, as well as their proliferation, contributing to an increased activation and leading to more effective anti-tumor responses. Finally, adoptive transfer of CD8+ T-cells in which PlxnA4 was knocked-out, was sufficient for an effective anti-tumor response.

In view of the above, the invention in one aspect relates to compounds capable of inhibiting plexin-A2 and/or plexin-A4, wherein said compounds are specifically targeting plexin-A2 and/or plexin-A4 on or in CD8-positive (CD8+) T-cells. Such compound inhibiting plexin-A2 and/or plexin-A4 include compounds comprising a polypeptide, a polypeptidic agent, or an aptamer binding to plexin-A2 and/or plexin-A4; compounds which are inducing degradation of plexin-A2 and/or of plexin-A4; or compounds which are interfering with expression of plexin-A2 and/or of plexin-A4. In particular, such compounds are selected from a polypeptide comprising an immunoglobulin variable domain, an antibody or a fragment thereof, an alpha-body, a nanobody, an intrabody, an aptamer, a DARPin, an affibody, an affitin, an anticalin, a monobody, a bicyclic peptide, a PROTAC, or a LYTAC; or is chosen from any combination of any of the foregoing; wherein the compounds are binding plexin-A2 and/or plexin-A4. With “specifically targeting plexin-A2 and/or plexin-A4 on or in CD8-positive (CD8+) T-cells” is meant that the intended compound is preferentially or selectively binding to plexin-A2 and/or plexin-A4 on or in CD8+ T-cells compared to binding to plexin-A2 and/or plexin-A4 present on or in cells different from CD8+ T-cells. Such preferential or selective binding may be achieved by e.g. the compound having a higher affinity for binding to plexin-A2 and/or plexin-A4 on or in CD8+ T-cells compared to binding to plexin-A2 and/or plexin-A4 present on or in cells different from CD8+ T-cells. Such preferential or selective binding may alternatively be achieved by targeting the plexin-A2 and/or plexin-A4-binding/inhibiting moiety to CD8+ T-cells. As such, any of the compounds capable of inhibiting plexin-A2 and/or plexin-A4, wherein said compounds are specifically targeting plexin-A2 and/or plexin-A4 on or in CD8-positive (CD8+) T-cells, may further comprise a moiety binding to a CD8+ T-cell-specific surface marker different from plexin-A2 and/or plexin-A4; in particular the CD8+ T-cell-specific surface marker different from plexin-A2 and/or plexin-A4 is CD8 or CD69.

In one embodiment, any of the above compounds is specifically targeting plexin-A2 and/or plexin-A4 on or in CD8+ T-cells in a tumor and/or in the tumor micro-environment of a subject having a tumor. The specificity for a tumor and/or the tumor-environment can in one embodiment be achieved by means of intra- or peri-tumoral administration of the compounds of the invention. Alternatively, the compounds of the invention can be delivered by means of a carrier, wherein the cargo of the carrier is the compound inhibiting plexin-A2 and/or plexin-A4, wherein the carrier is targeting its cargo to the tumor and/or tumor micro-environment, and/or wherein release of the cargo from the carrier can be controlled to occur in the tumor and/or in the tumor micro-environment. Such carriers include for instance viruses, oncolytic viruses, cells adoptively transferred to the subject, or exosomes, nanoparticles or microbubbles.

Before explaining further aspects of the invention, terms as used hereinabove are clarified.

### Plexins

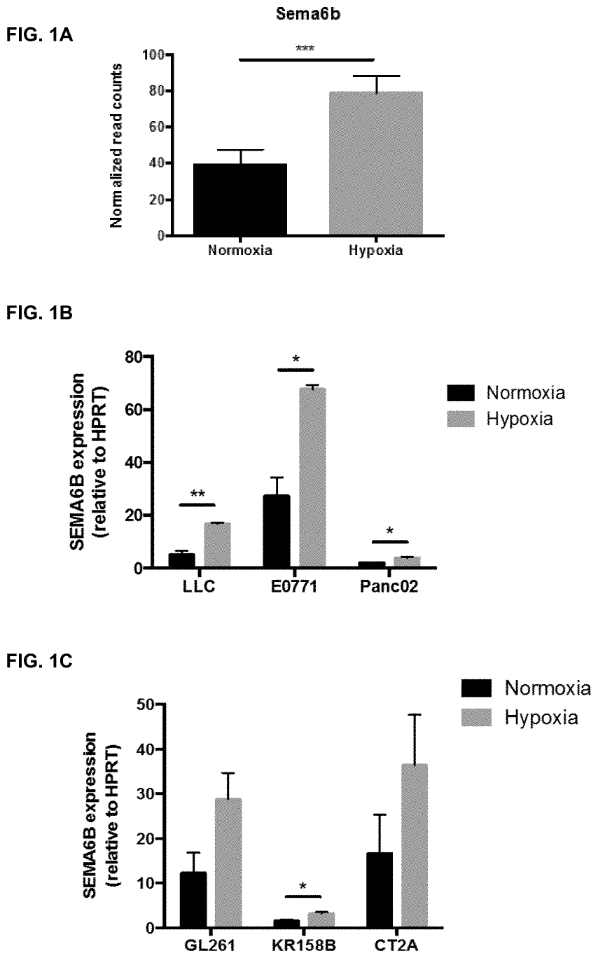

Plexins are membrane proteins known as being involved in semaphorin (Sema) signaling, a process that involves co-receptors such as neuropilins (Nrps) as well as receptor tyrosine kinases (RTKs) such as VEGFR2, Met, ErbB2 and off-track (OTK). Semaphorins are involved in regulating morphology and motility of a plethora of cell types. Four classes (A to D) of plexins are known in vertebrates (Alto & Terman 2017, Methods Mol Biol 1493:1-25). As shown in FIG. 1 of Alto & Terman 2017, and in its generality, interactions of plexins-A2 and -A4 have been reported with semaphorins of classes 3 and 6 (Sema3, Sema6) in vertebrates, as well of plexin-A4 with semaphorins of class 5 (Sema5) in invertebrates. Further in its generality, the interaction of plexins-A2 and -A4 with the soluble Sema3 can further involve neuropilins-1 and -2, RTKs, integrins, B7-H4, proteoglycans and cell adhesion molecules as (co-)receptors. Likewise, the interaction of plexins-A2 and -A4 with Sema6 can further involve RTKs and TREM2 (triggering receptor expressed on myeloid cells 2) as (co-)receptors; whereas the interaction of plexins-A4 with Sema5 (in invertebrates) can further involve neuropilin-2, RTKs, and proteoglycans (FIG. 1 of Alto & Terman 2017, Methods Mol Biol 1493:1-25). When zooming in on the semaphorins, plexins-A2 and -A4 appear to interact with Sema3A, Sema3D, Sema6A and Sema6B; in different plexin complexes, plexin-A4 further appears to interact with Sema3C, Sema3F, Sema6D and (invertebrate) Sema5B (Table 1 of Hota & Buck 2012, Cell Mol Life Sci 69:3765-3805; Smolkin et al. 2018, J Cell Sci 131:jcs208298; Kang et al. 2018, Nat Immunol 19:561-570).

Plexin-A2: the GeneCards Human Genome Database provides “Plexin A2”, “Semaphorin Receptor OCT”, “Transmembrane Protein OCT”, “Plexin 2”, “PLXN2”, “OCT”, and “KIAA0463” as aliases for PLXNA2. The GenBank reference PLXNA2 mRNA sequence is known under accession no. NM_025179.4.

Plexin-A4: the GeneCards Human Genome Database provides “Plexin A4”, “PLXNA4A”, “PLXNA4B”, “Epididymis Secretory Sperm Binding Protein”, “FAYV2820”, “PRO34003”, “KIAA1550”, and “PLEXA4” as aliases for PLXNA4. The GenBank reference PLXNA4 mRNA sequences are known under accession nos. NM_001105543.1, NM_020911.1, and NM_181775.3.

### Functional Plexin-A2 and Functional Plexin-A4

Functional plexin-A2 or plexin-A4, when referred to herein, is defined as plexin-A2 or plexin-A4 that is expressed and to which no “foreign” (in the sense of non-naturally occurring, artificially made, man-made, or any combination thereof) compound such as pharmacological inhibitor is bound or linked, wherein the “foreign” compound is capable of interfering directly (e.g. competing) or indirectly (e.g. by inducing degradation of plexin-A2 or plexin-A4) with the binding of plexin-A2 or plexin-A4 with any one of its potential natural binding partners (see hereinabove; and further including for instance, binding to itself in case of homo-(di-)merization, or to one-another in case of hetero-(di-)merization). Functional plexin-A2 or plexin-A4 may be exposed on the surface of CD8+ T-cells, or may be stored inside CD8+ T-cells such as stored in a manner allowing quick release to the cell surface.

As such, functional plexin-A2 or functional plexin-A4 can be lacking on and/or in a cell by repressing, inhibiting, or blocking expression of plexin-A2 or plexin-A4, or by binding of a “foreign” compound (as meant hereinabove) to plexin-A2 or plexin-A4. In particular, functional plexin-A2 and/or plexin-A4 is lacking, or is substantially lacking, on and/or in the isolated CD8+ T-cells of the invention.

Genetic modification of CD8+ T-cells isolated from a subject is one means of forcing the CD8+ T-cells to lack functional plexin-A2 and/or plexin-A4. Such genetic modification can be aimed at repressing, reducing, or inhibiting ongoing expression of plexin-A2 and/or plexin-A4 in the isolated (unmodified) CD8+ T-cells, and/or can be aimed at preventing or inhibiting de novo expression of plexin-A2 and/or plexin-A4, e.g. in case expression of plexin-A2 and/or plexin-A4 is low or non-existing in the isolated (unmodified) CD8+ T-cells.

Shielding (part of the) plexin-A2 and/or plexin-A4 protein exposed on the surface of CD8+ T-cells and/or stored within CD8+ T-cells by means of contacting the CD8+ T-cells with a pharmacological inhibitor of plexin-A2 and/or plexin-A4 is another means of causing CD8+ T-cells to lack functional plexin-A2 and/or plexin-A4. The said shielding can be envisaged as neutralizing (part of the) plexin-A2 and/or plexin-A4 protein for interaction with other (natural) binding partners. Such pharmacological inhibitors per se are known in the art, see e.g. WO 2012/114339 and WO 2015/037009 as discussed in the Background section, and alternatives are discussed in more detail hereinafter. In particular, such pharmacological inhibitors bind to plexin-A2 and/or plexin-A4 with high specificity and/or, optionally, with high affinity. Concurrent binding of a single inhibitor to both plexin-A2 and plexin-A4 is not excluded as such inhibitor can be bispecific.

Plexin-A2 and/or plexin-A4 protein present inside CD8+ T-cells or on the surface of CD8+ T-cells can further be the target of pharmacologic knock-down such as by molecules or agents inducing specific proteolytic degradation of plexin-A2 and/or plexin-A4 protein.

The agent causing CD8+ T-cell to (substantially) lack functional plexin-A2 and/or plexin-A4 or causing neutralization of plexin-A2 and/or of plexin-A4 as referred to herein may be part of a larger molecule further comprising a moiety directing the agent to CD8+ T-cells.

### Polypeptide, Polypeptidic Molecule or Agent

A polypeptide in general is a molecule comprising at least one polypeptidic bond (condensation of non-side chain carboxyl of one amino acid and non-side chain amino group of second amino acid) between two adjacent amino acids. A polypeptidic agent in general is (i) a molecule comprising at least one polypeptidic bond between two adjacent amino acids wherein one of the amino acids cannot be incorporated in the polypeptidic agent by means of translation/biological production in a cell (which can also be referred to as non-natural amino acid), (ii) a molecule comprising at least one non-natural polypeptidic bond (between natural amino acids, between a natural amino acid and a non-natural amino acid, or between two non-natural amino acids), or (iii) a molecule comprising at least one polypeptidic bond between two adjacent amino acids (between natural amino acids, between a natural amino acid and a non-natural amino acid, or between two non-natural amino acids) and further comprising a non-peptidic moiety.

Polypeptides or polypeptidic molecules may comprise intramolecular disulfide bonds, or may be connected to other polypeptides or polypeptidic molecules by e.g. intermolecular disulfide bonds. Synthesis of a polypeptide or polypeptidic molecule may be synthetic. Standard protein chemistry may be used to introduce an activatable N- or C-terminus. Alternatively additions may be made by fragment condensation or native chemical ligation e.g. as described in (Dawson et al. 1994, Science 266:776-779), or by enzymes, for example using subtiligase (Chang et al. 1994, PNAS 91:12544-8; Hikari et al. 2008, Bioorg Med Chem Lett 18:6000-6003). Alternatively, the peptides may be extended or modified by further conjugation through disulphide bonds. This has the additional advantage of allowing the first and second peptide to dissociate from each other once within the reducing environment of the cell. Furthermore, addition of e.g. drugs or other moieties may be accomplished in the same manner, using appropriate chemistry, coupling at the N- or C-termini or via side chains. Suitably the coupling is conducted in such a manner that it does not block the activity of either entity. The unnatural amino acids incorporated into peptides and proteins may include 1) a ketone functional group (as found in para or meta acetyl-phenylalanine) that can be specifically reacted with hydrazines, hydroxylamines and their derivatives (Wang et al. 2003, PNAS 100:56-61; Zeng et al. 2006, Bioorg Med Chem Lett 16:5356-5359), 2) azides (as found in p-azido-phenylalanine) that can be reacted with alkynes via copper catalysed “click chemistry” or strain promoted (3+2) cyloadditions to form the corresponding triazoles (Chin et al. 2002, J Am Chem Soc 124:9026-7; Deiters et al. 2003, J Am Chem Soc 125:11782-3), or azides that can be reacted with aryl phosphines, via a Staudinger ligation (Tsao et al. 2005, Chembiochem 6:2147-9), to form the corresponding amides, 4) alkynes that can be reacted with azides to form the corresponding triazole (Deiters & Schultz 2005, Bioorg Med Chem Lett 15:1521-4), 5) boronic acids (boronates) than can be specifically reacted with compounds containing more than one appropriately spaced hydroxyl group or undergo palladium mediated coupling with halogenated compounds (Brustad et al. 2008, Angew Chem Int Ed Engl 47:8220-3), 6) metal chelating amino acids, including those bearing bipyridyls, that can specifically co-ordinate a metal ion (Xie et al. 2007, Angew Chem Int Ed Engl 46:9239-42).

Unnatural amino acids may be incorporated into proteins and peptides displayed on phage by transforming E. coli with plasmids or combinations of plasmids bearing: 1) the orthogonal aminoacyl-tRNA synthetase and tRNA that direct the incorporation of the unnatural amino acid in response to a codon, 2) the phage DNA or phagemid plasmid altered to contain the selected codon at the site of unnatural amino acid incorporation (Liu et al. 2008, PNAS 105:17688-93; Tian et al. 2004, J Am Chem Soc 126(49): 15962-3). The orthogonal aminoacyl-tRNA synthetase and tRNA may be derived from the Methancoccus janaschii tyrosyl pair or a synthetase (Chin et al. 2002, PNAS 99:11020-4) and tRNA pair that naturally incorporates pyrrolysine (Yanagisawa et al. 2008, Chem Biol 15:1187-97; Neumann et al. 2008, Nat Chem Biol 4:232-4). The codon for incorporation may be the amber codon (UAG) another stop codon (UGA, or UAA), alternatively it may be a four-base codon. The aminoacyl-tRNA synthetase and tRNA may be produced from existing vectors, including the pBK series of vectors, pSUP (Ryu & Schultz 2006, Nat Methods 3:263-5) vectors and pDULE vectors (Farell et al. 2005, Nat Methods 2:377-84). The E. coli strain used will express the F′ pilus (generally via a tra operon). When amber suppression is used the E. coli strain will not itself contain an active amber suppressor tRNA gene. The amino acid will be added to the growth media, preferably at a final concentration of 1 mM or greater. Efficiency of amino acid incorporation may be -enhanced by using an expression construct with an orthogonal ribosome binding site and translating the gene with ribo-X (Wang et al. 2007, Nat Biotechnol 25:770-7). This may allow efficient multi-site incorporation of the unnatural amino acid.

Non-natural amino acids include D-amino acids (although some can be incorporated into peptidic molecules by some bacteria); N-methyl or N-alkyl amino acids; constrained amino acid side chains such as proline analogues, bulky side-chains, Calpha-substituted derivatives (e.g. a simple derivative is Aib (2-aminoisobutyric acid), H2N—C(CH3)2-COOH); and cyclo amino acids (a simple derivative being amino-cyclopropylcarboxylic acid).

Non-natural peptidic bond or peptide surrogate bonds include N-alkylation (CO—NR), reduced peptide bonds (CH2-NH—), peptoids (N-alkyl amino acids, NR—CH2-CO), thio-amides (CS-NH), azapeptides (CO—NH—NR), trans-alkene (RHC═C—), retro-inverso (NH—CO), urea surrogates (NH—CO—NHR). The peptide backbone length may also be modulated, i.e. β^{2,3}-amino acids, (NH—CR—CH2-CO, NH—CH2-CHR—CO); or backbone conformation may be constrained by e.g. substitutions on the alpha-carbon on amino acids (e.g. Aib). Non-peptidic moiety, in general, is any moiety different from an amino acid (natural or non-natural) or modification introduced to obtain a non-natural peptidic bond or altered backbone length. Non-peptidic moieties include e.g. capping or blocking groups, polyethyleneglycol (PEG) groups, drugs, molecular scaffolding moieties and the like.

Pharmacologic Inhibition of Plexin-A2 and/or Plexin-A4

Hereinafter different types of molecules that can be designed to inhibit Plexin-A2 or Plexin-A4 are described. Combinations of different molecules (of the same type or from a different type) each individually inhibiting either Plexin-A2 or Plexin-A4 are possible, thus creating a Plexin-A2- and Plexin-A4-inhibiting mixture of molecules. Intramolecular combinations of different molecules (of the same type or from a different type) each individually inhibiting either Plexin-A2 or Plexin-A4 are also possible, thus creating a Plexin-A2- and Plexin-A4-inhibiting molecules.

Pharmacological inhibition in general occurs by means of an agent inhibiting plexin-A2 and/or plexin-A4. In particular, such pharmacological inhibitor is binding, such as specifically binding to plexin-A2 and/or to plexin-A4. Such binding may occur with high affinity although this is not an absolute requirement. Likewise possible, but not absolutely required, such binding may induce internalization of plexin-A2 and/or plexin-A4. The pharmacological inhibitor of plexin-A2 and/or plexin-A4 may for instance have a binding affinity (dissociation constant) to (one of) its target of about 1000 nM or less, a binding affinity of about 100 nM or less, a binding affinity of about 50 nM or less, a binding affinity of about 10 nM or less, or a binding affinity of about 1 nM or less. Alternatively, the pharmacological inhibitor of plexin-A2 and/or plexin-A4 may exert the desired level of inhibition of plexin-A2 and/or of plexin-A4 with an IC50 of 1000 nM or less, with an IC50 of 500 nM or less, with an IC50 of 100 nM or less, with an IC50 of 50 nM or less, with an IC50 of 10 nM or less, or with an IC50 of 1 nM or less.

In general, the agent inhibiting plexin-A2 and/or plexin-A4 is a polypeptide, a polypeptidic agent, an aptamer, or a combination of any of the foregoing. Examples of such pharmacologic inhibitors or agents inhibiting plexin-A2 and/or plexin-A4 include immunoglobulin variable domains, antibodies or a fragment thereof, alpha-bodies, nanobodies, intrabodies, aptamers, DARPins, affibodies, affitins, anticalins, monobodies, and bicyclic peptides—when selected and screened for with care, each of these agents is known to exert excellent binding specificity to its target.

Inhibition of plexin-A2 and/or plexin-A4 can for example refer to inhibition of binding of ligands to plexin-A2 and/or to plexin-A4 (such as determinable in an assay comprising isolated plexin-A2 and/or isolated plexin-A4 protein (or isolated parts of such proteins such as isolated soluble parts of such proteins); or such as determinable in an assay relying on plexin-A2 and/or plexin-A4 expressed, such as recombinantly expressed, in or on a cell, on a phage, . . . ), or can for example refer to functional inhibition (such as determinable in cell proliferation or cell migration assays). The Examples provided hereinafter applied some of these assays.

The term “antibody” as used herein, refers to an immunoglobulin (Ig) molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The term “immunoglobulin domain” as used herein refers to a globular region of an antibody chain (such as e.g., a chain of a conventional 4-chain antibody or a chain of a heavy chain antibody), or to a polypeptide that essentially consists of such a globular region. Immunoglobulin domains are characterized in that they retain the immunoglobulin fold characteristic of antibody molecules, which consists of a two-layer sandwich of about seven antiparallel β-strands arranged in two β-sheets, optionally stabilized by a conserved disulphide bond.

The specificity of an antibody/immunoglobulin/immunoglobulin variable domain (IVD) for an antigen is defined by the composition of the antigen-binding domains in the antibody/immunoglobulin/IVD (usually one or more of the CDRs, the particular amino acids of the antibody/immunoglobulin/IVD interacting with the antigen forming the paratope) and the composition of the antigen (the parts of the antigen interacting with the antibody/immunoglobulin/IVD forming the epitope). Specificity of binding is understood to refer to a binding between an antibody/immunoglobulin/IVD with a single target molecule or with a limited number of target molecules that (happen to) share an epitope recognized by the antibody/immunoglobulin/IVD.

Affinity of an antibody/immunoglobulin/IVD for its target is a measure for the strength of interaction between an epitope on the target (antigen) and an epitope/antigen binding site in the antibody/immunoglobulin/IVD. It can be defined as:

$${K}_{A} = \frac{[ Ab - Ag ]}{[ Ab ] [ Ag ]}$$

Wherein KA is the affinity constant, [Ab] is the molar concentration of unoccupied binding sites on the antibody/immunoglobulin/IVD, [Ag] is the molar concentration of unoccupied binding sites on the antigen, and [Ab-Ag] is the molar concentration of the antibody-antigen complex.

Avidity provides information on the overall strength of an antibody/immunoglobulin/IVD-antigen complex, and generally depends on the above-described affinity, the valency of antibody/immunoglobulin/IVD and of antigen, and the structural interaction of the binding partners. The term “immunoglobulin variable domain” (abbreviated as “IVD”) as used herein means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Methods for delineating/confining a CDR in an antibody/immunoglobulin/IVD have been described in the art (and include the Kabat, Chothia, IMTG, Martin, Gelfand, and Honneger systems; see Dondelinger et al. 2018, Front Immunol 9:2278).

The term “immunoglobulin single variable domain” (abbreviated as “ISVD”), equivalent to the term “single variable domain”, defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associated) immunoglobulin domains such as light and heavy chain variable domains, i.e., by a VH-VL pair of immunoglobulin domains, which jointly bind to an epitope of the respective antigen. In contrast, immunoglobulin single variable domains are capable of specifically binding to an epitope of the antigen without pairing with an additional immunoglobulin variable domain. The binding site of an immunoglobulin single variable domain is formed by a single VH/VHH or VL domain. Hence, the antigen binding site of an immunoglobulin single variable domain is formed by no more than three CDRs. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody® (as defined herein, and including but not limited to a VHH); other single variable domains, or any suitable fragment of any one thereof. In particular, the immunoglobulin single variable domain may be a Nanobody® (as defined herein) or a suitable fragment thereof. Note: Nanobody®, Nanobodies® and Nanoclone® are registered trademarks of Ablynx N.V. For a general description of Nanobodies®, reference is made to the further description below, as well as to the prior art cited herein, such as e.g. described in WO2008/020079.

“VHH domains”, also known as VHHs, VHH domains, VHH antibody fragments, and VHH antibodies, have originally been described as the antigen binding immunoglobulin (variable) domain of “heavy chain antibodies” (i.e., of “antibodies devoid of light chains”; Hamers-Casterman et al (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody®, reference is made to the review article by Muyldermans (Reviews in Molecular Biotechnology 74: 277-302, 2001), as well as to the following patent applications, which are mentioned as general background art: WO 94/04678, WO 95/04079, WO 96/34103; WO 94/25591, WO 99/37681, WO 00/40968, WO 00/43507, WO 00/65057, WO 01/40310, WO 01/44301, EP 1134231 and WO 02/48193; WO 97/49805, WO 01/21817, WO 03/035694, WO 03/054016 and WO 03/055527; WO 03/050531; WO 01/90190; WO 03/025020; WO 04/041867, WO 04/041862, WO 04/041865, WO 04/041863, WO 04/062551, WO 05/044858, WO 06/40153, WO 06/079372, WO 06/122786, WO 06/122787 and WO 06/122825. As described in these references, Nanobody® (in particular VHH sequences and partially humanized Nanobody®) can in particular be characterized by the presence of one or more “Hallmark residues” in one or more of the framework sequences. A further description of the Nanobody®, including humanization and/or camelization of Nanobody®, as well as other modifications, parts or fragments, derivatives or “Nanobody® fusions”, multivalent constructs (including some non-limiting examples of linker sequences) and different modifications to increase the half-life of the Nanobody® and their preparations can be found e.g. in WO 08/101985 and WO 08/142164.

“Domain antibodies”, also known as “Dabs” (the terms “Domain Antibodies” and “dAbs” being used as trademarks by the GlaxoSmithKline group of companies) have been described in e.g., EP 0368684, Ward et al. (Nature 341: 544-546, 1989), Holt et al. (Tends in Biotechnology 21: 484-490, 2003) and WO 03/002609 as well as for example WO 04/068820, WO 06/030220, and WO 06/003388. Domain antibodies essentially correspond to the VH or VL domains of non-camelid mammalians, in particular human 4-chain antibodies. In order to bind an epitope as a single antigen binding domain, i.e., without being paired with a VL or VH domain, respectively, specific selection for such antigen binding properties is required, e.g. by using libraries of human single VH or VL domain sequences. Domain antibodies have, like VHHs, a molecular weight of approximately 13 to approximately 16 kDa and, if derived from fully human sequences, do not require humanization for e.g. therapeutic use in humans. It should also be noted that single variable domains can be derived from certain species of shark (for example, the so-called “IgNAR domains”, see for example WO 05/18629).

Immunoglobulin single variable domains such as Domain antibodies and Nanobody® (including VHH domains and humanized VHH domains), can be subjected to affinity maturation by introducing one or more alterations in the amino acid sequence of one or more CDRs, which alterations result in an improved affinity of the resulting immunoglobulin single variable domain for its respective antigen, as compared to the respective parent molecule. Affinity-matured immunoglobulin single variable domain molecules of the invention may be prepared by methods known in the art, for example, as described by Marks et al. (Biotechnology 10:779-783, 1992), Barbas, et al. (Proc. Nat. Acad. Sci, USA 91: 3809-3813, 1994), Shier et al. (Gene 169: 147-155, 1995), Yelton et al. (Immunol. 155: 1994-2004, 1995), Jackson et al. (J. Immunol. 154: 3310-9, 1995), Hawkins et al. (J. Mol. Biol. 226: 889 896, 1992), Johnson and Hawkins (Affinity maturation of antibodies using phage display, Oxford University Press, 1996). The process of designing/selecting and/or preparing a polypeptide, starting from an immunoglobulin single variable domain such as a Domain antibody or a Nanobody®, is also referred to herein as “formatting” said immunoglobulin single variable domain; and an immunoglobulin single variable domain that is made part of a polypeptide is said to be “formatted” or to be “in the format of” said polypeptide. Examples of ways in which an immunoglobulin single variable domain can be formatted and examples of such formats for instance to avoid glycosylation will be clear to the skilled person based on the disclosure herein. Immunoglobulin single variable domains such as Domain antibodies and Nanobody® (including VHH domains) can be subjected to humanization, i.e. increase the degree of sequence identity with the closest human germline sequence. In particular, humanized immunoglobulin single variable domains, such as Nanobody® (including VHH domains) may be immunoglobulin single variable domains that are as generally defined for in the previous paragraphs, but in which at least one amino acid residue is present (and in particular, at least one framework residue) that is and/or that corresponds to a humanizing substitution (as defined herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized.

Alphabodies are also known as Cell-Penetrating Alphabodies and are small 10 kDa proteins engineered to bind to a variety of antigens.

Aptamers have been selected against small molecules, toxins, peptides, proteins, viruses, bacteria, and even against whole cells. DNA/RNA/XNA aptamers are single stranded and typically around 15-60 nucleotides in length although longer sequences of 220 nt have been selected; they can contain non-natural nucleotides (XNA) as described for antisense RNA. A nucleotide aptamer binding to the vascular endothelial growth factor (VEGF) was approved by FDA for treatment of macular degeneration. Variants of RNA aptamers are spiegelmers are composed entirely of an unnatural L-ribonucleic acid backbone. A Spiegelmer of the same sequence has the same binding properties of the corresponding RNA aptamer, except it binds to the mirror image of its target molecule.

Peptide aptamers consist of one (or more) short variable peptide domains, attached at both ends to a protein scaffold, e.g. the Affimer scaffold based on the cystatin protein fold.

A further variation is described in e.g. WO 2004/077062 wherein e.g. 2 peptide loops are attached to an organic scaffold to arrive at a bicyclic peptide (which can be further multimerized). Phage-display screening of such peptides yielding bicyclic peptides binding with high-affinity to a target has proven to be possible in e.g. WO 2009/098450.

DARPins stands for designed ankyrin repeat proteins. DARPin libraries with randomized potential target interaction residues, with diversities of over 10\hat{ }12 variants, have been generated at the DNA level. From these, DARPins can be selected for binding to a target of choice with picomolar affinity and specificity. Affitins, or nanofitins, are artificial proteins structurally derived from the DNA binding protein Sac7d, found in Sulfolobus acidocaldarius. By randomizing the amino acids on the binding surface of Sac7d and subjecting the resulting protein library to rounds of ribosome display, the affinity can be directed towards various targets, such as peptides, proteins, viruses, and bacteria.

Anticalins are derived from human lipocalins which are a family of naturally binding proteins and mutation of amino acids at the binding site allows for changing the affinity and selectivity towards a target of interest. They have better tissue penetration than antibodies and are stable at temperatures up to 70° C.

Monobodies are synthetic binding proteins that are constructed starting from the fibronectin type III domain (FN3) as a molecular scaffold.

Affibodies are composed of alpha helices and lack disulfide bridges and are based on the Z or IgG-binding domain scaffold of protein A wherein amino acids located in the parental binding domain are randomized. Screening for affibodies binding to a desired target typically is performed using phage display.

Intrabodies are antibodies binding and/or acting to intracellular target; this typically requires the expression of the antibody within the target cell, which can be accomplished by gene therapy/genetic modification.

Pharmacologic Knock-Down of Plexin-A2 and/or Plexin-A4

Several technologies can be applied to cause pharmacologic knock-down of plexin-A2 and/or plexin-A4. Outlined hereafter are the general principles of agents causing pharmacologic knock-down of a target protein by means of inducing (proteolytic) degradation of that target protein.

A proteolysis targeting chimera, or PROTAC, is a chimeric polypeptidic molecule comprising a moiety recognized by an ubiquitin ligase and a moiety binding to a target protein. Interaction of the PROTAC with the target protein causes it to be poly-ubiquinated followed by proteolytic degradation by a cell's own proteasome. As such, a PROTAC provides the possibility of pharmacologically knocking down a target protein. The moiety binding to a target protein can be a peptide or a small molecule (reviewed in, e.g., Zou et al. 2019, Cell Biochem Funct 37:21-30). Other such target protein degradation inducing technologies include dTAG (degradation tag; see, e.g., Nabet et al. 2018, Nat Chem Biol 14:431), Trim-Away (Clift et al. 2017, Cell 171:1692-1706), chaperone-mediated autophagy targeting (Fan et al. 2014, Nat Neurosci 17:471-480) and SNIPER (specific and non-genetic inhibitor of apoptosis protein (IAP)-dependent protein erasers; Naito et al. 2019, Drug Discov Today Technol, doi:10.1016/j.ddtec.2018.12.002).

Lysosome targeting chimeras, or LYTACs, are chimeric molecules comprising a moiety binding to a lysosomal targeting receptor (LTR) and a moiety binding to a target protein (such as an antibody). Interaction of the LYTAC with the target protein causes it to be internalized followed by lysosomal degradation. A prototypic LTR is the cation-independent mannose-6-phosphate receptor (ciMPR) and an LTR binding moiety is e.g. an agonist glycopeptide ligand of ciMPR. The target protein can be a secreted protein or a membrane protein (see, e.g., Banik et al. 2019, doi.org/10.26434/chemrxiv.7927061.v1).

Genetic Inhibition of Plexin-A2 and/or of Plexin-A4

Genetic inhibition of plexin-A2 and/or plexin-A4 in the context of the current invention can be obtained by fairly standard technologies of which some are detailed hereinafter.

Downregulating expression of a gene encoding a target is feasible through gene therapy (e.g., by administering siRNA, shRNA or antisense oligonucleotides to the target gene) and through gene therapeutic antagonists include such entities as antisense oligonucleotides, gapmers, siRNA,shRNA, zinc-finger nucleases, meganucleases, Argonaute, TAL effector nucleases, CRISPR-Cas effectors, and nucleic acid aptamers.

One process of modulating/downregulating expression of a gene of interest relies on antisense oligonucleotides (ASOs), or variants thereof such as gapmers. An antisense oligonucleotide (ASO) is a short strand of nucleotides and/or nucleotide analogues that hybridizes with the complementary mRNA in a sequence-specific manner via Watson-Crick base pairing. Formation of the ASO-mRNA complex ultimately results in downregulation of target protein expression (Chan et al. 2006, Clin Exp Pharmacol Physiol 33:533-540; this reference also describes some of the software available for assisting in design of ASOs). Modifications to ASOs can be introduced at one or more levels: phosphate linkage modification (e.g. introduction of one or more of phosphodiester, phosphoramidate or phosphorothioate bonds), sugar modification (e.g. introduction of one or more of LNA (locked nucleic acids), 2′-O-methyl, 2′-O-methoxy-ethyl, 2′-fluoro, S-constrained ethyl or tricyclo-DNA and/or non-ribose modifications (e.g. introduction of one or more of phosphorodiamidate morpholinos or peptide nucleic acids). The introduction of 2′-modifications has been shown to enhance safety and pharmacologic properties of antisense oligonucleotides. Antisense strategies relying on degradation of mRNA by RNase H requires the presence of nucleotides with a free 2′-oxygen, i.e. not all nucleotides in the antisense molecule should be 2′-modified. The gapmer strategy has been developed to this end. A gapmer antisense oligonucleotide consists of a central DNA region (usually a minimum of 7 or 8 nucleotides) with (usually 2 or 3) 2′-modified nucleosides flanking both ends of the central DNA region. This is sufficient for the protection against exonucleases while allowing RNAseH to act on the (2′-modification free) gap region. Antidote strategies are available as demonstrated by administration of an oligonucleotide fully complementary to the antisense oligonucleotide (Crosby et al. 2015, Nucleic Acid Ther 25:297-305).

Another process to modulate expression of a gene of interest is based on the natural process of RNA interference. It relies on double-stranded RNA (dsRNA) that is cut by an enzyme called Dicer, resulting in double stranded small interfering RNA (siRNA) molecules which are 20-25 nucleotides long. siRNA then binds to the cellular RNA-Induced Silencing Complex (RISC) separating the two strands into the passenger and guide strand. While the passenger strand is degraded, RISC is cleaving mRNA specifically at a site instructed by the guide strand. Destruction of the mRNA prevents production of the protein of interest and the gene is ‘silenced’. siRNAs are dsRNAs with 2 nt 3′ end overhangs whereas shRNAs are dsRNAs that contains a loop structure that is processed to siRNA. shRNAs are introduced into the nuclei of target cells using a vector (e.g. bacterial or viral) that optionally can stably integrate into the genome. Apart from checking for lack of cross-reactivity with non-target genes, manufacturers of RNAi products provide guidelines for designing siRNA/shRNA. siRNA sequences between 19-29 nt are generally the most effective. Sequences longer than 30 nt can result in nonspecific silencing. Ideal sites to target include AA dinucleotides and the 19 nt 3′ of them in the target mRNA sequence. Typically, siRNAs with 3′ dUdU or dTdT dinucleotide overhangs are more effective. Other dinucleotide overhangs could maintain activity but GG overhangs should be avoided. Also to be avoided are siRNA designs with a 4-6 poly(T) tract (acting as a termination signal for RNA pol III), and the G/C content is advised to be between 35-55%. shRNAs should comprise sense and antisense sequences (advised to each be 19-21 nt in length) separated by loop structure, and a 3′ AAAA overhang. Effective loop structures are suggested to be 3-9 nt in length. It is suggested to follow the sense-loop-antisense order in designing the shRNA cassette and to avoid 5′ overhangs in the shRNA construct. shRNAs are usually transcribed from vectors, e.g. driven by the Pol III U6 promoter or H1 promoter. Vectors allow for inducible shRNA expression, e.g. relying on the Tet-on and Tet-off inducible systems commercially available, or on a modified U6 promoter that is induced by the insect hormone ecdysone. A Cre-Lox recombination system has been used to achieve controlled expression in mice. Synthetic shRNAs can be chemically modified to affect their activity and stability. Plasmid DNA or dsRNA can be delivered to a cell by means of transfection (lipid transfection, cationic polymer-based nanoparticles, lipid or cell-penetrating peptide conjugation) or electroporation. Viral vectors include lentiviral, retroviral, adenoviral and adeno-associated viral vectors.

Ribozymes (ribonucleic acid enzymes) are another type of molecules that can be used to modulate expression of a target gene. They are RNA molecules capable of catalyzing specific biochemical reactions, in the current context capable of targeted cleavage of nucleotide sequences. Examples of ribozymes include the hammerhead ribozyme, the Varkud Satellite ribozyme, Leadzyme and the hairpin ribozyme. Besides the use of the inhibitory RNA technology, modulation of expression of a gene of interest can be achieved at DNA level such as by gene therapy to knock-out or disrupt the target gene. As used herein, a “gene knock-out” can be a gene knockdown or the gene can be knocked out by a mutation such as, a point mutation, an insertion, a deletion, a frameshift, or a missense mutation by techniques such as described hereafter, including, but not limited to, retroviral gene transfer. Another way in which genes can be knocked out is by the use of zinc finger nucleases. Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of the endogenous DNA repair machinery, these reagents can be used to precisely alter the genomes of higher organisms. Other technologies for genome customization that can be used to knock out genes are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes). Another recent genome editing technology is the CRISPR/Cas system, which can be used to achieve RNA-guided genome engineering. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway. Recently, it was demonstrated that the CRISPR-Cas editing system can also be used to target RNA. It has been shown that the Class 2 type VI-A CRISPR-Cas effector C2c2 can be programmed to cleave single stranded RNA targets carrying complementary protospacers (Abudayyeh et al. 2016 Science353/science.aaf5573). C2c2 is a single-effector endoRNase mediating ssRNA cleavage once it has been guided by a single crRNA guide toward the target RNA.

Methods for administering nucleic acids include methods applying non-viral (DNA or RNA) or viral nucleic acids (DNA or RNA viral vectors). Methods for non-viral gene therapy include the injection of naked DNA (circular or linear), electroporation, the gene gun, sonoporation, magnetofection, the use of oligonucleotides, lipoplexes (e.g. complexes of nucleic acid with DOTAP or DOPE or combinations thereof, complexes with other cationic lipids), dendrimers, viral-like particles, inorganic nanoparticles, hydrodynamic delivery, photochemical internalization (Berg et al. 2010, Methods Mol Biol 635:133-145) or combinations thereof.

Many different vectors have been used in human nucleic acid therapy trials and a listing can be found on http://www.abedia.com/wileyvectors.php. Currently the major groups are adenovirus or adeno-associated virus vectors (in about 21% and 7% of the clinical trials), retrovirus vectors (about 19% of clinical trials), naked or plasmid DNA (about 17% of clinical trials), and lentivirus vectors (about 6% of clinical trials). Combinations are also possible, e.g. naked or plasmid DNA combined with adenovirus, or RNA combined with naked or plasmid DNA to list just a few. Other viruses (e.g. alphaviruses, vaccinia viruses such as vaccinia virus Ankara) are used in nucleic acid therapy and are not excluded in the context of the current invention.

Administration may be aided by specific formulation of the nucleic acid e.g. in liposomes (lipoplexes) or polymersomes (synthetic variants of liposomes), as polyplexes (nucleic acid complexed with polymers), carried on dendrimers, in inorganic (nano)particles (e.g. containing iron oxide in case of magnetofection), or combined with a cell penetrating peptide (CPP) to increase cellular uptake. Organ- or cellular-targeting strategies may also be applied to the nucleic acid (nucleic acid combined with organ- or cell-targeting moiety); these include passive targeting (mostly achieved by adapted formulation) or active targeting (e.g. by coupling a nucleic acid-comprising nanoparticle with any compound (e.g. an aptamer or antibody or antigen binding molecule) binding to a target organ- or cell-specific antigen) (e.g. Steichen et al. 2013, Eur J Pharm Sci 48:416-427).

CPPs enable translocation of the drug of interest coupled to them across the plasma membrane. CPPs are alternatively termed Protein Transduction Domains (TPDs), usually comprise 30 or less (e.g. 5 to 30, or 5 to 20) amino acids, and usually are rich in basic residues, and are derived from naturally occurring CPPs (usually longer than 20 amino acids), or are the result of modelling or design. A non-limiting selection of CPPs includes the TAT peptide (derived from HIV-1 Tat protein), penetratin (derived from Drosophila Antennapedia—Antp), pVEC (derived from murine vascular endothelial cadherin), signal-sequence based peptides or membrane translocating sequences, model amphipathic peptide (MAP), transportan, MPG, polyarginines; more information on these peptides can be found in Torchilin 2008 (Adv Drug Deliv Rev 60:548-558) and references cited therein. CPPs can be coupled to carriers such as nanoparticles, liposomes, micelles, or generally any hydrophobic particle. Coupling can be by absorption or chemical bonding, such as via a spacer between the CPP and the carrier. To increase target specificity an antibody binding to a target-specific antigen can further be coupled to the carrier (Torchilin 2008, Adv Drug Deliv Rev 60:548-558). CPPs have already been used to deliver payloads as diverse as plasmid DNA, oligonucleotides, siRNA, peptide nucleic acids (PNA), proteins and peptides, small molecules and nanoparticles inside the cell (Stalmans et al. 2013, PloS One 8:e71752).

Any other modification of the DNA or RNA to enhance efficacy of nucleic acid therapy is likewise envisaged to be useful in the context of the applications of the nucleic acid or nucleic acid comprising compound as outlined herein. The enhanced efficacy can reside in enhanced expression, enhanced delivery properties, enhanced stability and the like. The applications of the nucleic acid or nucleic acid comprising compound as outlined herein may thus rely on using a modified nucleic acid as described above. Further modifications of the nucleic acid may include those suppressing inflammatory responses (hypoinflammatory nucleic acids).

### Targeting CD8+ T-Cells

It was demonstrated that e.g. anti-CD8+F(ab′)2-conjugated nanoparticles specifically bound to CD8+ T-cells. Likewise, an anti-PD1-conjugated nanoparticle specifically bound to the subset of CD8+ T-cells expressing PD1. Therapeutic cargo with these nanoparticles was demonstrated as well (Schmid et al. 2017, Nat Commun 8:1747).

Targeting activated T-cells is feasible by means of targeting CD69, one of the earliest markers during activation of T-cells (Ziegler et al. 1994, Stem cells 12:456-465).

In view thereof, any molecule or carrier (such as nanoparticles, viruses, cells, microbubbles) can be targeted to CD8+ T-cells by means of their coupling or association with a moiety specifically binding to e.g. CD8 or CD69. To ensure such specific binding, the CD8+ T-cell targeting can for instance be by means of immunoglobulin variable domains, antibodies or a fragment thereof, alpha-bodies, nanobodies, intrabodies, aptamers, DARPins, affibodies, affitins, anticalins, monobodies, and bicyclic peptides—when selected and screened for with care, each of these agents is known to exert excellent binding specificity to its target. Bispecific antibodies binding to both CD8 and Plexin-A4 were demonstrated to retain binding capacity to both CD8 and Plexin-A4 on cells, as described hereinafter in the Examples.

### Adoptive Cell Transfer

In general, adoptive cell transfer (also known as cellular adoptive immunotherapy or T-cell transfer therapy) refers to the administration of ex-vivo expanded T cells to a subject in need of such adoptive cell transfer, wherein the original T cell is obtained from the subject prior to its expansion. The ex-vivo expanded T cells can, prior to their transfer back in the subject, be genetically modified. Well-known genetic modifications include genetic engineering such as to cause the T-cells to express antitumor T cell receptors (TCRs) or chimeric antigen receptors (CARs) to increase anti-tumor activity of the transferred T cells. It can also be envisaged, i.e., to engineer the cells produced for adoptive cell transfer to express an inhibitor of Plexin-A2 and/or of Plexin-A4, or to load expanded cells with an inhibitor of Plexin-A2 and/or of Plexin-A4 prior to adoptive transfer. As T-cells are known to produce exosomes, such exosomes would be, together with their cargo, delivered in the tumor or in the tumor micro-environment. In the context of the present invention, T-cells could be forced to themselves lack or to substantially lack functional plexin-A2 and/or plexin-A4 (by means of genetic modification, a pharmacological inhibitor, or a pharmacological knock-down agent). In particular, in view of the experimental data presented herein, it is plausible that omission of plexin-A2 and/or plexin-A4 in T-cells employed in TCR-engineered or CAR-engineered adoptive T-cell transfer will further increase their anti-tumor activity, which could be further enhanced by the cells expanded for adoptive transfer to express an inhibitor of plexin-A2 and/or plexin-A4.

### Oncolytic Virus

Oncolytic viruses are viruses preferentially targeting tumor cells (compared to healthy cells) and causing lysis of tumor cells, therewith releasing new viruses or virions that can target other tumor cells. The oncolytic virus-mediated lysis of tumor cells is also thought to boost the immune system of the subject having the tumor. Specificity of an oncolytic viruses towards a tumor can be obtained by transductional targeting (via a viral coat protein targeting the tumor) and/or via non-transductional targeting. The latter can involve transcriptional targeting (such as expression of the required viral and/or other genes under the control of tumor-specific promoter) or viral replication can be controlled by means of micro-RNAs (miRNAs) or miRNA-response elements (MREs) as expression of miRNAs in tumors often differs from that in healthy cells. Oncolytic viruses may also carry a payload not strictly required for viral replication, known examples include expression of a single-chain anti-VEGF antibody (Frentzen et al. 2009, PNAS 106:12915-12920), of mAb (whole monoclonal antibody), Fab (antigen-binding fragment) and scFv (single chain variable fragment) formats of an PD-1 binder (Kleinpeter et al. 2016, Oncoimmunol 5:e1220467), of intratumoral antibodies against vascular endothelial growth factor (VEGF), epidermal growth factor receptor (EGFR), and fibroblast activation protein (FAP) (Huang et al. 2015, Mol Ther Oncolytics 2:15003), and of an enhancer of replication (e.g. inhibitor of growth 4 (Ing4) enhancing replication of at least oncolytic HSV1716 in tumor cells; Conner et al. 2012, Cancer Gene Ther 19:499-507). Such modified oncolytic viruses are also termed armed oncolytic viruses (see, e.g., review of Bauzon & Hermiston 2014, Front Immunol 5:74). In this context, it can be plausibly envisaged to design an oncolytic virus expressing an inhibitor of Plexin-A2 and/or of Plexin-A4. The tumor-specificity of the oncolytic virus per se, and/or the tumor-specific expression of such inhibitor of Plexin-A2 and/or of Plexin-A4 endow such oncolytic virus with the competence as tumor-specific carrier. Types of viruses employed in the field of oncolytic cancer therapy include, but are not limited to: adenoviruses, vaccinia viruses, herpes viruses, reoviruses, measles viruses, and Newcastle disease viruses (N DV).

### Exosome Therapy

A relatively new and emerging therapeutic tool is relying on exosomes. Exosomes are normally shed from cells and are of endocytic origin. Exosomes from dendritic cells are involved in priming T-cells and natural killer (NK) cells. Exosomes from effector T-cells can carry cytotoxic activity. Exosomes from tumor cells may contain molecules involved in metastasis and/or invasion (see, e.g., Gao & Jiang 2018, Am J Cancer Res 8:2165-2175). Exosomes can be produced by cells expressing e.g. a tumor-targeting moiety and/or CD8+ T-cell-targeting moiety as well as an inhibitor of Plexin-A2 and/or of Plexin-A4. It may also become feasible to e.g. load exosomes produced by cells expressing e.g. a tumor-targeting moiety and/or CD8+ T-cell-targeting moiety, with an inhibitor of Plexin-A2 and/or of Plexin-A4. The resulting exosomes can then be subsequently administered to a subject having a tumor or cancer. Such exosomes therewith are tumor cell targeting and/or CD8+ T-cell-targeting carriers of an inhibitor of Plexin-A2 and/or of Plexin-A4. Tumor-specificity of such CD8+ T-cell-targeting exosomes can be enhanced by intra- or peri-tumoral administration(s) of these exosomes.

### Tumor-, or CD8+ T-Cell-Targeted Nanoparticles

Administration may be aided by specific formulation of the nucleic acid e.g. in liposomes (lipoplexes) or polymersomes (synthetic variants of liposomes), as polyplexes (nucleic acid complexed with polymers), carried on dendrimers, in inorganic (nano)particles (e.g. containing iron oxide in case of magnetofection), or combined with a cell penetrating peptide (CPP) to increase cellular uptake. Tumor-, cancer- or neoplasm-targeting strategies may also be applied to the nucleic acid (nucleic acid combined with tumor-, cancer-, or neoplasm-targeting moiety); these include passive targeting (mostly achieved by adapted formulation) or active targeting (e.g. by coupling a nucleic acid-comprising nanoparticle with folate or transferrin, or with an aptamer or antibody binding to an target cell-specific antigen) (e.g. Steichen et al. 2013, Eur J Pharm Sci 48:416-427).

CPPs enable translocation of the drug of interest coupled to them across the plasma membrane. CPPs are alternatively termed Protein Transduction Domains (TPDs), usually comprise 30 or less (e.g. 5 to 30, or 5 to 20) amino acids, and usually are rich in basic residues, and are derived from naturally occurring CPPs (usually longer than 20 amino acids), or are the result of modelling or design. A non-limiting selection of CPPs includes the TAT peptide (derived from HIV-1 Tat protein), penetratin (derived from Drosophila Antennapedia—Antp), pVEC (derived from murine vascular endothelial cadherin), signal sequence based peptides or membrane translocating sequences, model amphipathic peptide (MAP), transportan, MPG, polyarginines; more information on these peptides can be found in Torchilin 2008 (Adv Drug Deliv Rev 60:548-558) and references cited therein. CPPs can be coupled to carriers such as nanoparticles, liposomes, micelles, or generally any hydrophobic particle. Coupling can be by absorption or chemical bonding, such as via a spacer between the CPP and the carrier. To increase target specificity an antibody binding to a target-specific antigen can further be coupled to the carrier (Torchilin 2008, Adv Drug Deliv Rev 60:548-558). CPPs have already been used to deliver payloads as diverse as plasmid DNA, oligonucleotides, siRNA, peptide nucleic acids (PNA), proteins and peptides, small molecules and nanoparticles inside the cell (Stalmans et al. 2013, PloS One 8:e71752).

### Tumor-, or CD8+ T-Cell-Targeted Microbubbles

Using a phage screening procedure/screening of phage-displayed peptide libraries, Laakkonen et al. 2002 (Nat Med 8:751-755) identified the cyclic nonapeptide LyP-1 as targeting moiety to tumor cells, tumor lymphatics and tumor-associated macrophages (TAMs). Yan et al. 2012 (J Control Release 157:118-125) discuss nanoparticles modified with LyP-1 and demonstrated targeted delivery of drug-loaded LyP-1-conjugated and PEGylated liposomes having the same homing specificity as LyP-1. LyP-1 is binding to the p32/gC1q receptor present on these tumor-associated cells. The same nonapeptide was earlier used to target to a tumor microbubbles. Such microbubbles are sensitive to ultrasound, i.e. ultrasound makes them burst. Ultrasonic tumor molecular imaging or drug-delivery and therapy was therewith in reach and combines targeted delivery of the microbubbles with targeted delivery of the cargo contained in the microbubble (Li et al. 2009, Proceedings of 31st Annual International Conference of the IEEE Eng Med Biol Sci Minneapolis, Minn., USA, Sep. 2-6, 2009; 463-466). Obviously, the above is only one example. The technique can be modified to target CD8+ T-cells and/or tumor-cells, and the cargo of such microbubbles can be envisaged to be an inhibitor of Plexin-A2 and/or of Plexin-A4. Such microbubbles therewith are tumor cell targeting and/or CD8+ T-cell-targeting carriers of an inhibitor of Plexin-A2 and/or of Plexin-A4. Tumor-specificity of such CD8+ T-cell-targeting microbubbles can be further enhanced by intra- or peri-tumoral administration(s) of these exosomes, although this is not strictly required as release of the cargo can be spatially controlled by the ultrasound administration.

### Pharmaceutical Compositions

A further aspect of the invention relates to pharmaceutical compositions comprising any of the above compounds inhibiting plexin-A2 and/or plexin-A4 according to the invention. In particular, such pharmaceutical composition comprises besides the compounds inhibiting plexin-A2 and/or plexin-A4 according to the invention a carrier which is pharmaceutically acceptable (which can be administered to a subject without in itself causing severe side effects) and suitable for supporting stability, and storage, if required. Such pharmaceutical composition can further comprise an anticancer agent (detailed further hereinafter, including chemotherapeutic agent, targeted therapy agent, and immunotherapeutic agent).

### Medical Use

The invention also envisages any of the compounds inhibiting plexin-A2 and/or plexin-A4 according to the invention, or any of the pharmaceutical compositions according to the invention to be suitable for use as medicament; for use in (a method of) treating, inhibiting, or suppressing a tumor or cancer; or for use in (a method of) treating, inhibiting, or suppressing a tumor or cancer, further in combination with surgery, radiation, chemotherapy, targeted therapy, immunotherapy, or a further anticancer agent; or for any of (i) use in the manufacture of a medicament, (ii) use in the manufacture of a medicament for treating, inhibiting, or suppressing a tumor or cancer, or (iii) use in the manufacture of a medicament for treating, inhibiting, or suppressing a tumor or cancer by further in combination with surgery, radiation, chemotherapy, targeted therapy, immunotherapy, or a further anticancer agent.

When for use in treating, inhibiting, or suppressing a tumor or cancer, any of (i) surgery, (ii) radiation, (iii) chemotherapy, (iv) targeted therapy, (v) immunotherapy, or (vi) a further anticancer agent may further be for use in combination with any of the compounds inhibiting plexin-A2 and/or plexin-A4 according to the invention, or with any of the pharmaceutical compositions according to the invention. Any of a chemotherapeutic agent, a targeted therapy agent, an immunotherapeutic agent, or an anticancer agent may be for use in the manufacture of a medicament for treating, inhibiting, or suppressing a tumor or cancer in combination with any of the compounds inhibiting plexin-A2 and/or plexin-A4 according to the invention, or with any of the pharmaceutical compositions according to the invention.

Further medical uses include methods of treating, inhibiting, or suppressing a tumor or cancer in a subject having a tumor or cancer, said methods comprising the step of administering (in particular: administering a therapeutically effective dose of any of the compounds inhibiting plexin-A2 and/or plexin-A4 according to the invention, or with any of the pharmaceutical compositions according to the invention. Such methods may further comprise (simultaneous, separate or sequential) combination with administration of any of (i) surgery, (ii) radiation, (iii) chemotherapy, (iv) targeted therapy, (v) immunotherapy, or (vi) a further anticancer agent.

Further medical uses include methods of treating, inhibiting, or suppressing a tumor or cancer in a subject having a tumor or cancer, said methods comprising the step of administering (in particular: administering a therapeutically effective dose of) any of (i) surgery, (ii) radiation, (iii) chemotherapy, (iv) targeted therapy, (v) immunotherapy, or (vi) an anticancer agent, further in combination with of any of the compounds inhibiting plexin-A2 and/or plexin-A4 according to the invention, or with any of the pharmaceutical compositions according to the invention.

Before moving to kits of the invention, some of terms as used in the medical use section are explained in more detail.

### Treatment/Therapeutically Effective Amount

“Treatment”/“treating” refers to any rate of reduction, delaying or retardation of the progress of the disease or disorder, or a single symptom thereof, compared to the progress or expected progress of the disease or disorder, or singe symptom thereof, when left untreated. This implies that a therapeutic modality on its own may not result in a complete or partial response (or may even not result in any response), but may, in particular when combined with other therapeutic modalities, contribute to a complete or partial response (e.g. by rendering the disease or disorder more sensitive to therapy). More desirable, the treatment results in no/zero progress of the disease or disorder, or singe symptom thereof (i.e. “inhibition” or “inhibition of progression”), or even in any rate of regression of the already developed disease or disorder, or singe symptom thereof. “Suppression/suppressing” can in this context be used as alternative for “treatment/treating”. Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. by patient well-being. In the case of quantitative evaluation, the significant amelioration is typically a 10% or more, a 20% or more, a 25% or more, a 30% or more, a 40% or more, a 50% or more, a 60% or more, a 70% or more, a 75% or more, a 80% or more, a 95% or more, or a 100% improvement over the situation prior to treatment. The timeframe over which the improvement is evaluated will depend on the type of criteria/disease observed and can be determined by the person skilled in the art.

A “therapeutically effective amount” refers to an amount of a therapeutic agent to treat or prevent a disease or disorder in a subject (such as a mammal). In the case of cancers, the therapeutically effective amount of the therapeutic agent may reduce the number of cancer cells; reduce the primary tumor size; inhibit (i.e., slow down to some extent and preferably stop) cancer cell infiltration into peripheral organs; inhibit (i.e., slow down to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, e.g., be measured by assessing the duration of survival (e.g. overall survival), time to disease progression (TTP), response rates (e.g., complete response and partial response, stable disease), length of progression-free survival, duration of response, and/or quality of life.

The term “effective amount” refers to the dosing regimen of the agent (e.g. antagonist as described herein) or composition comprising the agent (e.g. medicament or pharmaceutical composition). The effective amount will generally depend on and/or will need adjustment to the mode of contacting or administration. The effective amount of the agent or composition comprising the agent is the amount required to obtain the desired clinical outcome or therapeutic effect without causing significant or unnecessary toxic effects (often expressed as maximum tolerable dose, MTD). To obtain or maintain the effective amount, the agent or composition comprising the agent may be administered as a single dose or in multiple doses. The effective amount may further vary depending on the severity of the condition that needs to be treated; this may depend on the overall health and physical condition of the subject or patient and usually the treating doctor's or physician's assessment will be required to establish what is the effective amount. The effective amount may further be obtained by a combination of different types of contacting or administration.

The aspects and embodiments described above in general may comprise the administration of one or more therapeutic compounds to a subject (such as a mammal) in need thereof, i.e., harboring a tumor, cancer or neoplasm in need of treatment. In general a (therapeutically) effective amount of (a) therapeutic compound(s) is administered to the mammal in need thereof in order to obtain the described clinical response(s).

“Administering” means any mode of contacting that results in interaction between an agent (e.g. a therapeutic compound) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

### Anticancer Agent

The term anticancer agent is construed herein broadly as any agent which is useful or applicable in the treatment of a tumor or cancer in a subject. Anticancer agents comprise chemotherapeutic agents (usually small molecules) such as alkylating antineoplastic agents, anti-metabolites, anti-microtubule agents, topoisomerase inhibitors, and cytotoxic agents. The term further includes biological anticancer agents and immunotherapeutic drugs (such as immune checkpoint inhibitors) which are usually more specifically targeting the tumor or cancer (targeted therapy).

Chemotherapeutic agents may be one of the following compounds, or a derivative or analog thereof: doxorubicin and analogues [such as N-(5,5-diacetoxypent-1-yl)doxorubicin: Farquhar et al. 1998, J Med Chem 41:965-972; epirubicin (4′-epidoxorubicin), 4′-deoxydoxorubicin (esorubicin), 4′-iodo-4′-deoxydoxorubicin, and 4′-O-methyldoxorubicin: Arcamone et al. 1987, Cancer Treatment Rev 14:159-161 & Giuliani et al. 1980, Cancer Res 40:4682-4687; DOX—F-PYR (pyrrolidine analog of DOX), DOX—F-PIP (piperidine analog of DOX), DOX-F-MOR (morpholine analog of DOX), DOX—F-PAZ (N-methylpiperazine analog of DOX), DOX—F-HEX (hexamehtyleneimine analog of DOX), oxazolinodoxorubicin (3′deamino-3′-N, 4′-O-methylidenodoxorubicin, O-DOX): Denel-Bobrowska et al. 2017, Life Sci 178:1-8)], daunorubicin (or daunomycin) and analogues thereof [such as idarubicin (4′-demethoxydaunorubicin): Arcamone et al. 1987, Cancer Treatment Rev 14:159-161; 4′-epidaunorubicin; analogues with a simplified core structure bound to the monosaccharide daunosamine, acosamine, or 4-amino-2,3,6-trideoxy-L-threo-hexopyranose: see compounds 8-13 in Fan et al. 2007, J Organic Chem 72:2917-2928], amrubicin, vinblastine, vincristine, calicheamicin, etoposide, etoposide phosphate, CC-1065 (Boger et al. 1995, Bioorg Med Chem 3:611-621), duocarmycins (such as duocarmycin A and duocarmycin SA; Boger et al. 1995, Proc Natl Acad Sci USA 92:3642-3649), the duocarmycin derivative KW-2189 (Kobayashi et al. 1994, Cancer Res 54:2404-2410), methotrexate, methopterin, aminopterin, dichloromethotrexate, docetaxel, paclitaxel, epithiolone, combretastatin, combretastatin A4 phosphate, dolastatin 10, dolastatin 10 analogues (such as auristatins, e.g. auristatin E, auristatin-PHE, monomethyl auristatin D, monomethyl auristatin E, monomethyl auristatin F; see e.g. Maderna et al. 2014, J Med Chem 57:10527-10534), dolastatin 11, dolastatin 15, topotecan, camptothecin, mitomycin C, porfiromycin, 5-fluorouracil, 6-mercaptopurine, fludarabine, tamoxifen, cytosine arabinoside, adenosine arabinoside, colchicine, halichondrin B, cisplatin, carboplatin, mitomycin C, bleomycin and analogues thereof (e.g. liblomycin, Takahashi et al. 1987, Cancer Treatment Rev 14:169-177), melphalan, chloroquine, cyclosporin A, and maytansine (and maytansinoids and analogues thereof such as analogues comprising a disulfide or thiol substituent: Widdison et al. 2006, J Med Chem 49:4392-4408; maytansin analogs DM1 and DM4). By derivative is intended a compound that results from reacting the named compound with another chemical moiety, and includes a pharmaceutically acceptable salt, acid, base, ester or ether of the named compound.

Other therapeutic agents or drugs include: vindesine, vinorelbine, 10-deacetyltaxol, 7-epi-taxol, baccatin III, 7-xylosyltaxol, isotaxel, ifosfamide, chloroaminophene, procarbazine, chlorambucil, thiophosphoramide, busulfan, dacarbazine (DTIC), geldanamycin, nitroso ureas, estramustine, BCNU, CCNU, fotemustine, streptonigrin, oxaliplatin, methotrexate, aminopterin, raltitrexed, gemcitabine, cladribine, clofarabine, pentostatin, hydroxyureas, irinotecan, topotecan, 9-dimethylaminomethyl-hydroxy-camptothecin hydrochloride, teniposide, amsacrine; mitoxantrone; L-canavanine, THP-adriamycin, idarubicin, rubidazone, pirarubicin, zorubicin, aclarubicin, epiadriamycin (4′epi-adriamycin or epirubicin), mitoxantrone, bleomycins, actinomycins including actinomycin D, streptozotocin, calicheamycin; L-asparaginase; hormones; pure inhibitors of aromatase; androgens, proteasome inhibitors; farnesyl-transferase inhibitors (FTI); epothilones; discodermolide; fostriecin; inhibitors of tyrosine kinases such as STI 571 (imatinib mesylate); receptor tyrosine kinase inhibitors such as erlotinib, sorafenib, vandetanib, canertinib, PKI 166, gefitinib, sunitinib, lapatinib, EKB-569; Bcr-Abl kinase inhibitors such as dasatinib, nilotinib, imatinib; aurora kinase inhibitors such as VX-680, CYC116, PHA-739358, SU-6668, JNJ-7706621, MLN8054, AZD-1152, PHA-680632; CDK inhibitors such as flavopirodol, seliciclib, E7070, BMS-387032; MEK inhibitors such as PD184352, U-0126; mTOR inhibitors such as CCI-779 or AP23573; kinesin spindle inhibitors such as ispinesib or MK-0731; RAF/MEK inhibitors such as Sorafenib, CHIR-265, PLX-4032, CI-1040, PD0325901 or ARRY-142886; bryostatin; L-779450; LY333531; endostatins; the HSP 90 binding agent geldanamycin, macrocyclic polyethers such as halichondrin B, eribulin, or an analogue or derivative of any thereof.

Monoclonal antibodies employed as anti-cancer agents include alemtuzumab (chronic lymphocytic leukemia), bevacizumab (colorectal cancer), cetuximab (colorectal cancer, head and neck cancer), denosumab (solid tumor's bony metastases), gemtuzumab (acute myelogenous leukemia), ipilumab (melanoma), ofatumumab (chronic lymphocytic leukemia), panitumumab (colorectal cancer), rituximab (Non-Hodgkin lymphoma), tositumomab (Non-Hodgkin lymphoma) and trastuzumab (breast cancer). Other antibodies include for instance abagovomab (ovarian cancer), adecatumumab (prostate and breast cancer), afutuzumab (lymphoma), amatuximab, apolizumab (hematological cancers), blinatumomab, cixutumumab (solid tumors), dacetuzumab (hematologic cancers), elotuzumab (multiple myeloma), farletuzumab (ovarian cancer), intetumumab (solid tumors), muatuzumab (colorectal, lung and stomach cancer), onartuzumab, parsatuzumab, pritumumab (brain cancer), tremelimumab, ublituximab, veltuzumab (non-Hodgkin's lymphoma), votumumab (colorectal tumors), zatuximab and anti-placental growth factor antibodies such as described in WO 2006/099698.

Immunotherapy is a promising new area of cancer therapeutics and several immunotherapies are being evaluated preclinically as well as in clinical trials and have demonstrated promising activity (Callahan et al. 2013, J Leukoc Biol 94:41-53; Page et al. 2014, Annu Rev Med 65:185-202). However, not all the patients are sensitive to immune checkpoint blockade and sometimes PD-1 or PD-L1 blocking antibodies accelerate tumor progression. To this purpose, combinatorial cancer treatments that include chemotherapies can achieve higher rates of disease control by impinging on distinct elements of tumor biology to obtain synergistic antitumor effects. It is now accepted that certain chemotherapies can increase tumor immunity by inducing immunogenic cell death and by promoting escape in cancer immunoediting. Immunotherapeutic agents include immune checkpoints antagonists including the cell surface protein cytotoxic T lymphocyte antigen-4 (CTLA-4) and programmed death-1 (PD-1) with their respective ligands. CTLA-4 binds to its co-receptor B7-1 (CD80) or B7-2 (CD86); PD-1 binds to its ligands PD-L1 (B7-H10) and PD-L2 (B7-DC).

Drug moieties known to induce immunogenic cell death include bleomycin, bortezomib, cyclophosphamide, doxorubicin, epirubicin, idarubicin, mafosfamide, mitoxantrone, oxaliplatin, and patupilone (Bezu et al. 2015, Front Immunol 6:187).

### Pharmaceutical Kits

A pharmaceutical kit refers in general to a packed pharmaceutical compound. Besides the one or more vials or containers comprising the pharmaceutical compound, such kits can comprise one or more vials of reconstitution fluid in case the pharmaceutical compound is provided as powder. A pharmaceutical kit in general also comprises a kit insert which, in case of an authorized medicine, itself also has been reviewed and approved by the health authorities (such as US FDA or EMEA). Thus, in a further aspect, the invention relates to pharmaceutical kits comprising as one component at least one of the compounds inhibiting plexin-A2 and/or plexin-A4 according to the invention, or at least one of the pharmaceutical compositions according to the invention.

Such pharmaceutical kits can optionally further comprise one or more anticancer agents.

### Tumor, Cancer, Neoplasm

The terms tumor (or tumour) and cancer are sometimes used interchangeably but can be distinguished from each other. A tumor refers to “a mass” which can be benign (more or less harmless) or malignant (cancerous). A cancer is a threatening type of tumor. A tumor is sometimes referred to as a neoplasm: an abnormal cell growth, usually faster compared to growth of normal cells. Benign tumors or neoplasms are nonmalignant/non-cancerous, are usually localized and usually do not spread/metastasize to other locations. Because of their size, they can affect neighboring organs and may therefore need removal and/or treatment. A cancer, malignant tumor or malignant neoplasm is cancerous in nature, can metastasize, and sometimes re-occurs at the site from which it was removed (relapse).

The initial site where a cancer starts to develop gives rise to the primary cancer. When cancer cells break away from the primary cancer (“seed”), they can move (via blood or lymph fluid) to another site even remote from the initial site. If the other site allows settlement and growth of these moving cancer cells, a new cancer, called secondary cancer, can emerge (“soil”). The process leading to secondary cancer is also termed metastasis, and secondary cancers are also termed metastases. For instance, liver cancer can arise as primary cancer, but can also be a secondary cancer originating from a primary breast cancer, bowel cancer or lung cancer; some types of cancer show an organ-specific pattern of metastasis.

Most cancer deaths are in fact caused by metastases, rather than by primary tumors (Chambers et al. 2002, Nature Rev Cancer 2:563-572).

### EXAMPLES

### Example 1. Materials and Methods

### Animals

Plxna4 KO mice on a C57BL/6 background were obtained from Dr. Castellani (Institut NeuroMyoGène, Université de Lyon, France). C57BL/6 mice were purchased from Charles River. OT-I mice were purchased from Taconic. All mice were used between 6 and 12 weeks old, without specific gender selection. In all experiments, littermate controls were used. Housing and all experimental animal procedures were approved by the Institutional Animal Care and Research Advisory Committee of the KU Leuven.

### Cell Lines

Murine Lewis lung carcinoma cells (LLC), B16-F10 melanoma cells, MC38 colon adenocarcinoma, and E0771 medullary breast adenocarcinoma (triple negative breast cancer, TNBC) cells were obtained from the American Type Culture Collection (ATCC). LLC-OVA and B16-F10-OVA cell lines were obtained by viral transduction with a pcDNA3-OVA plasmid. GL261-fluc glioma cells were a gift from U. Himmelreich (Biomedical MRI/MoSAIC, KU Leuven, Belgium). All cells were cultured in DMEM medium supplemented with 10% (heat-inactivated) Fetal Bovine Serum (FBS), 2 mM glutamine, 100 units/ml penicillin and 100 μg/ml streptomycin (All Gibco, Thermo Fisher Scientific) at 37° C. in a humidified atmosphere containing 5% CO₂.

### Bone Marrow Transplantation

Six-week-old C56BL/6 recipient mice were lethally irradiated with 9.5 Gy. Subsequently, 1×10² bone marrow cells from the appropriate genotype were injected intravenously (IV) via tail vein. Tumor experiments were initiated 6 to 8 weeks after bone marrow reconstitution. Red and white blood cell count was determined using a hemocytometer on peripheral blood. Also, flow cytometry analysis was carried out in blood collected in heparin with capillary pipettes by retro-orbital bleeding.

Syngeneic tumor models Adherent growing murine cells, 1×10⁶ LLC, 1×10⁶ MC38 and 1×10⁵ B16-F10 or B16-F10 OVA, were injected subcutaneously at the right side of the immunocompetent C57BL/6 mouse in a volume of 200 μl of PBS. Alternatively, 5×10⁵ E0771 medullary breast adenocarcinoma cell were injected orthotopically in the mammary fat pad of the second nipple on the right side in a volume of 50 ul of PBS. The E0771 model represents an immunologically “cold” breast cancer, and the immune infiltrate is dominated by immunosuppressive mo-MDSCs and M2-type TAMs. C57BL/6 mice bearing syngeneic tumors were randomized into groups (n=5) for treatment when tumor volumes reached 80 mm³. Tumor volumes were measured three times a week with a caliper and calculated using the formula: V=π×d²×D/6, where d is the minor tumor axis and D is the major tumor axis. At the end stage, tumors were weighted and collected for immunofluorescence and/or flow cytometric analyses.

### Orthotopic Glioma Tumor Model

For intracranial orthotopic syngeneic glioma model, a total of 1×10⁵ GL261-fluc cells were stereotactically injected in the brain striatum (2 mm right, 0.5 mm back, and 3 mm deep from the bregma) of WT→WT and Plxna4 KO→WT mice chimeras 7 weeks after re-constitution. Animal body weight was evaluated 3 times per week, and general behavior and symptomatology daily. Tumor volume was measured by bioluminescent imaging (BLI) at day 15. Therefore, mice were anesthetized with isoflurane and placed in an IVIS® 100 system (Perkin Elmer) and 126 mg/Kg of D-luciferin (Promega) was injected intraperitoneally (IP). Images were acquired 20 minutes after the injection and analyzed for maximum intensity of the photon flux by the living Image® 2.50.1 software (Perkin Elmer). Tumor volume was also addressed near to the end point by Magnetic Resonance Imaging (MRI) in a preclinical MR scanner (Bruker Biospec 94/20). At sacrifice, mice were perfused with saline followed by 2% paraformaldehyde (PFA) and brains were collected for immunofluorescence analyses, for which samples were fixed by immersion in 2% PFA and subsequently embedded in paraffin.

### CD8-Specific PlexinA2 Knockout Mice and Tumor Models

Conditional PlexinA2 (lox/lox) KO mouse line was intercrossed with CD8 specific CD8.CreERT2 mice (constitutive active Cre recombinase), for the specific deletion of PIxnA2 in CD8+ T cells. Adherent growing 1×10\hat{ }6 MC38 colon adenocarcinoma cells were injected subcutaneously at the right side of the mouse in a volume of 200 μl of PBS. Tumor volumes were measured three times a week with a caliper and calculated using the formula: V=p×d2×D/6, where d is the minor tumor axis and D is the major tumor axis. At the end stage, tumors were weighted. 5×10\hat{ }5 E0771 medullary breast adenocarcinoma cells were injected orthotopically in the mammary fat pad of the second nipple on the right side in a volume of 50 μl of PBS. Tumor volumes were measured three times a week with a caliper and calculated using the formula: V=p×d2×D/6, where d is the minor tumor axis and D is the major tumor axis. At the end stage, tumors were weighted and collected flow cytometric analyses.

### Histology and Immunostainings

Tumors and lymph nodes (LNs) were collected and fixed in 2% PFA for 24 hours, washed in 70% ethanol and embedded in paraffin. Serial sections were cut at 7 μm thickness with HM 355S automatic microtome (Thermo Fisher Scientific). Paraffin slides were first rehydrated to further proceed with antigen retrieval in Target Retrieval Solution, Citrate pH 6.1 (DAKO, Agilent). If necessary, 0.3% hydrogen peroxide was added to methanol, to block endogenous peroxidases.

Alternatively, lymph nodes (LNs) were collected in OCT compound (Leica) and frozen at −80° C. After cryo-sectioning (7 μm thickness), samples were thawed and washed with PBS once, followed by fixation with 4% PFA, for 10 minutes at room temperature. After 3 washed, endogenous peroxidases activity was blocked by incubating the sections in methanol containing 0.3% hydrogen peroxide. The sections were blocked with the appropriate serum (DAKO, Agilent; or e.g., 5% FBS and 5% rat serum) and incubated overnight with the following antibodies: rat anti-F4/80 (CI:A3-1, Serotec) 1:100, rabbit anti-Hypoxyprobe-1-Mab1 (Hypoxyprobe kit, Chem icon) 1:100, rat anti-CD34 (RAM34, BD Biosciences) 1:100, rat anti-CD31 (MEC 13.3, BD Biosciences) 1:50, rabbit anti-NG2 (Millipore) 1:200, rat anti-CD8 (4SM16, Thermo Fisher Scientific) 1:100, rat anti-PNAd (MECA-79, Biolegend) 1:100, or Biotin anti-mouse/human PNAd (MECA-79, Biolegend) 1:100. Hoechst 33342 solution (Thermo Fisher Scientific, 1:1000) was used to stain nuclei. Appropriate secondary antibodies were used: Alexa 488, 647 or 568 conjugated secondary antibodies (Molecular Probes), biotin-labeled antibodies (Jackson Immunoresearch) and, when necessary, TSA Plus Cyanine 3 and Cyanine 5 System amplification (Perkin Elmer, Life Sciences) were performed according to the manufacturer's instructions. Whenever sections were stained in fluorescence, ProLong Gold mounting medium without DAPI (Invitrogen) was used. Microscopic analysis was done with an Olympus BX41 microscope and CellSense imaging software.

### Hypoxia Assessment

Tumor hypoxia was detected by IP injection of 60 mg/kg pimonidazole hydrochloride into tumor-bearing mice 1 hour before the sacrifice. Mice were sacrificed and tumors were harvested. To detect the formation of pimonidazole adducts, tumor paraffin sections were immunostained with Hypoxyprobe-1-Mab1 (Hypoxyprobe kit, Chemicon) following the manufacturer's instructions.

### Blood Vessel Perfusion and Leakiness

Perfused tumor vessels were counted on tumor sections from mice injected IV with 50 μL of 0.05 mg FITC-conjugated lectin (Lycopersicon esculentum; Vector Laboratories) 10 minutes before the sacrifice. Tumors were collected in 2% PFA.

### Flow Cytometry

Tumor-bearing mice were sacrificed by cervical dislocation, and tumors, tumor-draining and non-draining LNs were harvested. Tumors were minced in aMEM medium (Lonza), containing Collagenase V

(Sigma), Collagenase D (Roche) and Dispase (Gibco), and incubated in the same solution for 30 minutes at 37° C. The digested tissue was filtered using a 70 μm pore sized mesh and cells were centrifuged 5 minutes at 300×g. LNs were processed on a 40 μm pore cell strainer in sterile PBS and cells were centrifuged for 10 minutes at 300×g. Blood samples were collected in heparin with capillary pipettes by retro-orbital bleeding. Red blood cell lysis was performed by using Hybri-Max™ (Sigma-Aldrich) or by using a home-made red blood cell lysis buffer (150 mM NH₄Cl, 0.1 mM EDTA, 10 mM KHCO₃, pH 7.4). Cells were resuspended in FACS buffer (PBS containing 2% FBS and 2 mM EDTA) and incubated for 15 minutes with Mouse BD Fc Block purified anti-mouse CD16/CD32 mAb (BD-Pharmingen) and stained for 30 minutes at 4° C. with: Fixable viability dye (eFluor™ 450 or eFluor™ 506, 1:500), anti-CD11b (M1/70, eFluor™ 506, 1:400), anti-F4/80 (BM8, Alexa Fluor 488, 1:200), anti-CD8 (53-6.7, APC or APC-Cγ7, 1:400; or Alexa Fluor™ 488, 1:100), anti-CD69 (H1.2F3, APC, 1:200), anti-IFN (XMG1.2, PE-Cγ7 1:100), anti-Gata3 (TWAJ, eFluor 660, 1:50), anti-T-bet (4610, PE-Cγ7, 1:40), anti-FOXP3 (FJK-16s, PerCP-Cγ5.5, 1:100) or anti-TCR V135.1/5.2 (MR9-4, APC, 1:200)-from Thermo Fisher Scientific; anti-CD45 (30-F11, APC-Cγ7, 1:300, or PerCP, 1:200), anti-CD115 (AFS98, PE-Cγ7, 1:200), anti-CD4 (RM4-5, PerCP-Cγ5.5 or APC-Cγ7, 1:400), anti-granzyme B (GB11, Alexa Fluor® 647, 1:100)—from BioLegend; and anti-TCR13 (H57-597, BV421, 1:300) and anti-Ly-6G (1A8, PE, 1:500)—from BD Biosciences. Cells were subsequently washed and resuspended in FACS buffer before FACS analysis or flow sorting by a FACS Verse, FACS Canto II or FACS Aria III (BD Biosciences), respectively. Data was analyzed by Flowio (TreeStar).

### T Cell Isolation and Activation

Naïve T cells were isolated from spleen, inguinal and axillary LNs. In brief, tissues were processed on a 40 μm pore cell strainer in sterile PBS and cells were centrifuged for 10 minutes at 300×g. Red blood cell lysis was performed using Hybri-Max™ (Sigma-Aldrich). Total splenocytes were cultured in T cell medium—RPMI medium supplemented with (heat-inactivated) 10% Fetal Bovine Serum (FBS), 100 units/ml penicillin and 100 μg/ml streptomycin, 1% MEM Non-Essential Amino Acids (NEAA), 25 μm beta-mercaptoethanol and 1 mM Sodium Pyruvate (all Gibco, Thermo Fisher Scientific)—at 37° C. in a humidified atmosphere containing 5% CO₂. According to the experimental requirements, T cells were activated for 3 days by adding CD3/CD28 Dynabeads (Thermo Fisher Scientific) at a bead-to-cell ratio of 1:1 and 30 U/ml rIL-2 (PeproTech).

At day 3 of activation, the beads were magnetically removed and activated T cells were further expanded for a maximum of 3 additional days in the presence of 30 U/ml rIL-2. To monitor cell proliferation, naïve T cells were labelled with 3.5 μM violet cell tracer (Thermo Fisher Scientific) at 37° C. for 20 minutes. The cells were subsequently washed with FACS buffer (PBS containing 2% FBS and 2 mM EDTA) and cultured according to the experimental requirements.

### T Cell Migration Assay

Migration of CD8⁺ cells was assessed by using transwell permeable supports with 5-μm polycarbonate membrane (Costar). CD8⁺ cells were isolated by using MagniSort Mouse CD8 T cell negative selection kit (eBioscience) according to the manufacturer's instructions. To determine cell migration in response to soluble factors, the lower chamber was pre-incubated with 0.1% FBS, 200 ng/ml CCL21 and 200 ng/ml CCL19 (all Peprotech) in T cell medium. CD8⁺ cells were incubated for 3 hours at 37° C. and migrated cells were collected and counted under the microscope. Alternatively, to determine cell migration in response to soluble factors, the lower chamber was pre-incubated with 0.1% FBS, 200 ng/ml CCL21, 200 ng/ml CCL19, 150 ng/ml CXCL9 and 50 ng/ml CXCL10 (all Peprotech) in T cell medium. CD8⁺ T cells were incubated for 2 (activated) or 3 hours (naïve) at 37° C. and migrated cells in the bottom chamber were collected and counted by FACS using Precision Count Beads' (Biolegend).

### T Cell Homing Assay

CD8⁺ T cells were isolated from WT and Plxna4 KO mice and were labelled with either 3.5 μM violet cell tracer (Thermo Fisher Scientific) or 1 μM carboxyfluorescein succinimidyl ester (CFSE; Thermo Fisher Scientific). Healthy C57BL/6 mice were injected IV with a 1:1 mixture between 1-2×10⁶ WT and KO T-cells. After 2 hours, lymph nodes (LNs) of the recipient mice were harvested. LNs were used for immunohistochemistry and flow cytometry to determine the percentage of WT and KO T-cells.

### Tumor Homing Assay

Activated WT and Plxna4 KO OT-I T cells were labelled with either 3.5 μM of Violet Cell Tracer or 1 μM of CFSE and injected intravenously with a 1:1 mixture between 2-3×10⁶ WT and Plxna4-deficient OT-I T cells into WT recipient mice with established B16-F10-OVA or LLC-OVA tumors. The tumors of recipient mice were harvested 24 and 48 hours after T cell transfer and analyzed by flow cytometry.

### Adoptive Cell Transfer

CD8⁺ T cells were isolated from transgenic Plxna4 WT/KO OT-I mice, generated by the intercross of Plxna4 heterozygous mice with OT-I positive mice in the host lab. These mice have a monoclonal population of naïve TCR transgenic CD8⁺ T cells (OT-I T cells) that recognize the immunodominant cytosolic chicken ovalbumin (OVA) “SIINFEKL” (SEQ ID NO:1) peptide. 1-2×10⁶ WT and Plxna4 KO OT-I T cells were injected into WT recipient mice carrying subcutaneous LLC-OVA tumors (8×10⁵ cells injected 5 days before T cell transfer).

Total splenocytes isolated from OT-1-PlexinA4 KO mice and littermate controls were activated with SIINFEKL (SEQ ID NO:1) peptide in the presence of IL-2. Six days later, CD8+ T cells were inoculated intravenously (2.5×10^{\hat{ }}6 cells per mouse) into recipient mice carrying subcutaneous B16-OVA tumors (1×10^{\hat{ }}5 cells injected 13 days before T cell transfer). For activation of OT-I T cells, total splenocytes from OT-I mice were isolated and cultured for 3 days in T cell medium with 1 μg/ml SIINFEKL (SEQ ID NO:1) peptide (IBA-LifeSciences) and 30 U/ml rIL-2 (PeproTech). At day 3 of activation, OT-I T cells were further expanded for a maximum of 3 additional days in the presence of 30 U/ml rIL-2. Recipient mice were treated with cyclophosphamide (100 mg/kg) 1 day before receiving effector CD8⁺ T cells and received daily i.p. injections of 5 ug of recombinant human IL-2 beginning the day of adoptive transfer and lasting for 4 days.

WT recipient mice carrying orthotopic B16-F10-OVA tumors (average tumor size of 30-50 mm³) were injected intravenously with either PBS, 2-3×10⁶ WT or the same number of Plxna4 KO OT-1 T cells. Recipient mice received daily intraperitoneal (IP) injections of 5 μg of recombinant human IL-2, beginning the day of adoptive transfer and lasting for 4 days. Tumor volume was measured at least 4 times per week and at the end of the experiment tumors were weighted and collected for flow cytometric analysis.

### Quantitative RT-PCR

RNA was extracted from sorted tumor-associated macrophages and tumor-infiltrating T cells with TRIzol (Life Technologies) according to the manufacturer's instructions. Reverse transcription to cDNA was performed with the SuperScript III First Strand cDNA Synthesis Kit (Life Technologies) according to the manufacturer's instructions. Pre-made assays were purchased from Applied Biosystem. cDNA, primer/probe mix and TaqMan Fast Universal PCR Master Mix were prepared in a volume of 10 μl according to manufacturer's instructions (Applied Biosystems). Samples were loaded into an optical 96-well Fast Thermal Cycling plate (Applied Biosystems) and qRT-PCR were performed using a QuantStudio 12K Flex Real-Time PCR System (Applied Biosystems). Samples were run in technical triplicates. Data was normalized to a housekeeping gene (HPRT) expression. The commercially available probes (Integrated DNA Technologies) used are listed in Table 1.

TABLE 1
List of probes used for gene expression analysis.

| Species | Gene | Exon location | Assay ID |
|---|---|---|---|
| Mouse | Hprt | Exon 2-3 | Mm.PT.58.32092191 |
| Mouse | PlxnA4 | Exon 2-3 | Mm.PT.58.8104978 |
| Human | Tbp | Exon 1-2 | Hs.PT.58v.39858774 |
| Human | PlxnA4 | Exon 29-30 | Hs.PT.58.4195119 |

### GTPase Pull Down Assay

Rac1 activation was measured by using a Rac1 activation assay kit (Thermo Fisher Scientific) according to the manufacturer's instructions. Briefly, fresh lysates of activated WT and PIxnA4 KO T cells (day 5/6 of activation) were incubated with the glutathione S-transferase (GST)-fused p21-binding domain of Pak1 (GST-Pak1-PBD, 20 rig) bound to glutathione resin at 4° C. for 60 minutes with gentle rocking. After being washed three times with lysis buffer, the samples were eluted in 2× SDS reducing sample buffer, and analyzed for bound Rac1 (GTP-Rac1) by western blot.

### Western Blotting

Protein concentration of cell extracts was determined by using Pierce™ bicinchoninic acid (BCA) reagent (Thermo Fisher Scientific) according to the manufacturer's instructions. Samples containing equivalent amounts of protein were subjected to 12% SDS-polyacrylamide gel electrophoresis. Proteins were transferred onto a nitrocellulose membrane using the Trans-Blot Turbo™ Transfer System (Bio-Rad) according to manufacturer's instructions. The membranes were blocked for non-specific binding in 5% non-fatty dry milk in Tris Buffered Saline-Tween 0.1% (50 mM Tris HCl ph 7.6, 150 mM NaCl, 0.1% Tween; TBS-T) for 1 hour at room temperature (RT) and incubated with primary antibody overnight (ON) at 4° C. The following antibodies were used: mouse anti-Rac1 (1:1000, Thermo Fisher Scientific). After incubation with the primary antibody, the membrane was washed for 15 minutes in TBS-T and incubated with the appropriate secondary antibody ( 1/5000 in 5% non-fatty dry milk in TBS-T) for 1 hours at RT. The following secondary antibodies were used: goat anti-mouse IgG-HRP (Santa Cruz biotechnology). The signal was visualized with Enhanced Chemiluminescent Reagents (ECL; Invitrogen) or SuperSignal™ West Femto Chemiluminescent Substrate (Thermo Fisher Scientific) with a digital imager (ImageQuant LAS 4000, GE Health Care Life Science Technologies). The results of the GTPase pull down assay were normalized against the corresponding band of the total proteins and quantified by densitometry.

### Statistics

Data entry and all analyses were performed in a blinded fashion. All statistical analyses were performed using GraphPad Prism software on mean values, calculated from the averages of technical replicates. Statistical significance was calculated by two-tailed unpaired t-test (or paired t-test in the case of in vivo homing assay) on two experimental conditions or two-way ANOVA when repeated measures were compared, with p<0.05 considered statistically significant. Survival curves were compared with the log-rank (Mantel-Cox) test. Statistical details of the experiments can be found in the figure legends. Detection of mathematical outliers was performed using the Grubbs' test in GraphPad. Sample sizes for all experiments were chosen based on previous experiences. Independent experiments were pooled and analyzed together whenever possible. All graphs show mean values±SEM.

### Example 2

### 2.1. Under Hypoxia Sema6B is Upregulated in Tumor-Associated Macrophages (TAMs) and in Tumor Cells

To analyse which chemoattractant/repulsive factors are implicated in the tumor microenvironment under hypoxia, transcriptional analysis was done in sorted TAMs from LLC tumors growing in wild-type mice and cultured under hypoxic and normoxic conditions. Sema6B was found to be specifically upregulated in TAMs cultured under hypoxic conditions (FIG. 1A). Previous studies showed that Sema3A was found to be strongly upregulated in cancer cells (reported in Casazza et al. 2013). In distinct tumor cell lines derived from solid tumors and GBM were grown under hypoxic conditions, also Sema6B was found to be specifically upregulated by hypoxia relative to normoxic conditions. (FIG. 1B). This indicates that hypoxic conditions of the tumor microenvironment might upregulate Sema6B in multiple cell types.

Class-6 semaphorins are single pass membrane bound semaphorins that were initially found to function as axon guidance factors, but that have recently been shown to be involved in other biological processes, including immune regulation. Plexin A4 (PIxA4) and PlexinA2 (PIxA2) transmembrane receptors both function as receptors for transmembrane class 6 semaphorins, as well as for secreted Sema3A in conjunction with Neuropilins as co-receptors. PlxnA4 appears to play a role in the regulation of the immune system in inflammation (Wen et al. 2010, J Exp Med 207:2943-2957; Yamamoto et al. 2008, Int Immunol 20, 413-420), but a role in immune-oncology is thus far unknown.

### 2.2. Plxna4 Expression is Dynamically Regulated in CD8⁺ T Lymphocytes

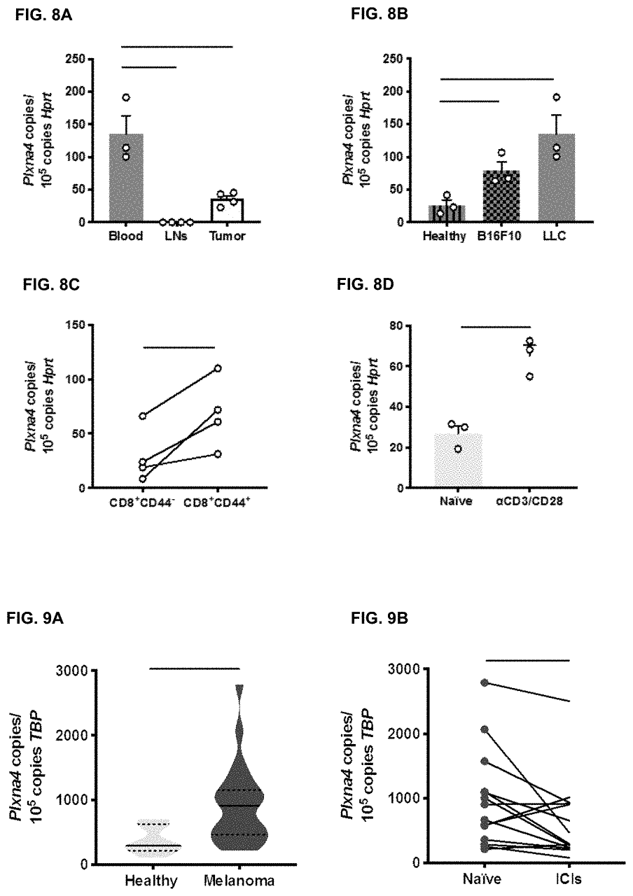

Cytotoxic T lymphocytes (CTLs) are among the most powerful anti-tumor cells in the immune system, and their infiltration level in the tumor microenvironment (TME) is correlated with good prognosis in several tumor types (for a review, e.g. Fridman et al. 2012, Nat Rev Cancer 12:298-306). PlxnA4 was described to play a role in the regulation of the immune system in sepsis (Wen et al. 2010, J Exp Med 207:2943-2957), and it seems to be a negative regulator of T-cell mediated immune responses (Yamamoto et al. 2008, Int Immunol 20:413-420). The role of PlxnA4 in CTLs in cancer context was studied as described herein. For that, we characterized the expression of Plxna4 in CTLs sorted from different organs of tumor-bearing mice. Plxna4 showed to be highly expressed in circulating CTLs, comparing to CTLs sorted from the lymph nodes (LNs) or the tumor bed (FIG. 8A). Interestingly, when we sorted circulating CTLs from healthy mice and compared their Plxna4 expression levels with their tumor-bearing counterparts, we found that Plxna4 is up-regulated in a tumor context (FIG. 8B), both in an orthotopic melanoma model (B16-F10 orthotopic injection) and in a subcutaneous lung model (based on LLC injection). These data suggested an involvement of PlxnA4 in CTL activation upon antigen presentation. Indeed, when comparing the expression of Plxna4 in antigen-experienced CD8+ T cells (CD44⁺) and their naïve counterparts (CD44⁻) in the circulation of tumor-bearing mice, we found that Plxna4 is up-regulated in antigen-experienced CTLs (FIG. 8C). Additionally, T-cell activation in vitro also showed to be sufficient for an induction of Plxna4 in spleen-derived CTLs (FIG. 8D). Taken together, these results suggest that Plxna4 expression becomes evident upon CTL activation but it is expressed at low levels in intratumoral CTLs.

Furthermore, in an attempt to translate these findings to human cancer, a cohort of melanoma patients was used. Similar to what was observed in murine models, CD8+ T cells in circulation of melanoma patients expressed higher levels of Plxna4, compared to CTLs isolated from the blood of healthy volunteers (FIG. 9A). Furthermore, when analyzing the expression of Plxna4 in these patients after one cycle of immunotherapy, a significant decrease of this molecule in CTLs from treated patients was observed, compared to the same patients before treatment (FIG. 9B). Interestingly, immunotherapy frequently leads to an increase in the proliferation of the pre-existing CTLs (Kamphorst et al. 2017, Proc Natl Acad Sci USA 114:4993-4998), a feature related to the observed phenotype of Plxna4 KO CTLs. For this reason, these observations support the blocking of PlxnA4 as an additional checkpoint inhibitor with potential use in the clinic.

### Example 3. Impaired Tumor Progression in Global PlxnA4 Knock-Out Mice

To assess the potential role of PlxnA4 in the stromal tumor microenvironment, the growth of distinct syngeneic tumor models was monitored in PlxnA4 knockout mice. Compared to wild-type (WT) controls mice, Plxna4 knockout (KO) mice (Yaron et al. 2005, Neuron 45:513-523) were phenotypically normal and had similar blood counts (Table 21; and Yamamoto et al. 2008, Int Immunol 20, 413-420). Subcutaneous LLC lung carcinomas and B16-F10 melanomas grew significantly slower in Plxna4 KO mice comparing to WT controls (Figures2A-2D).

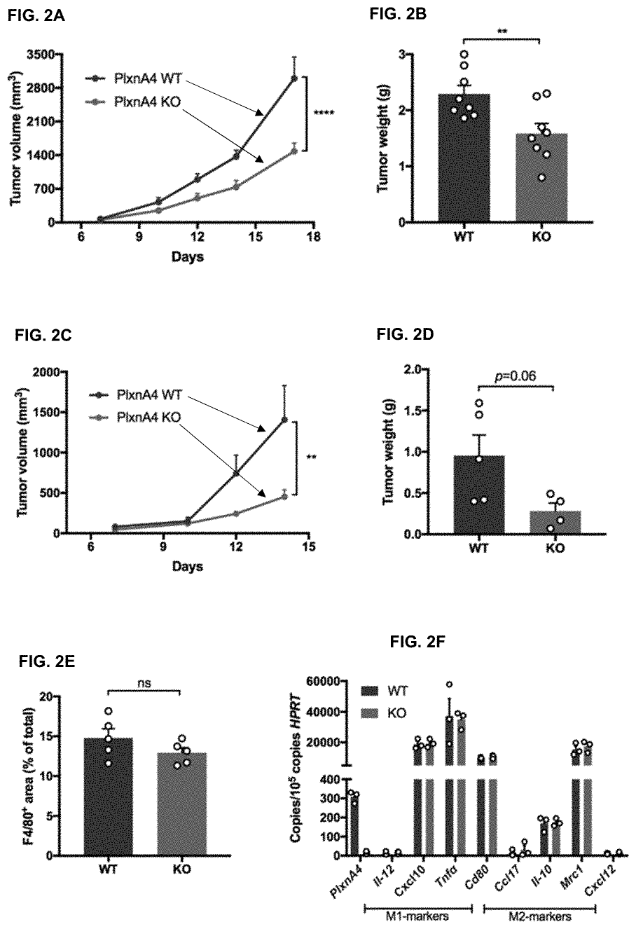

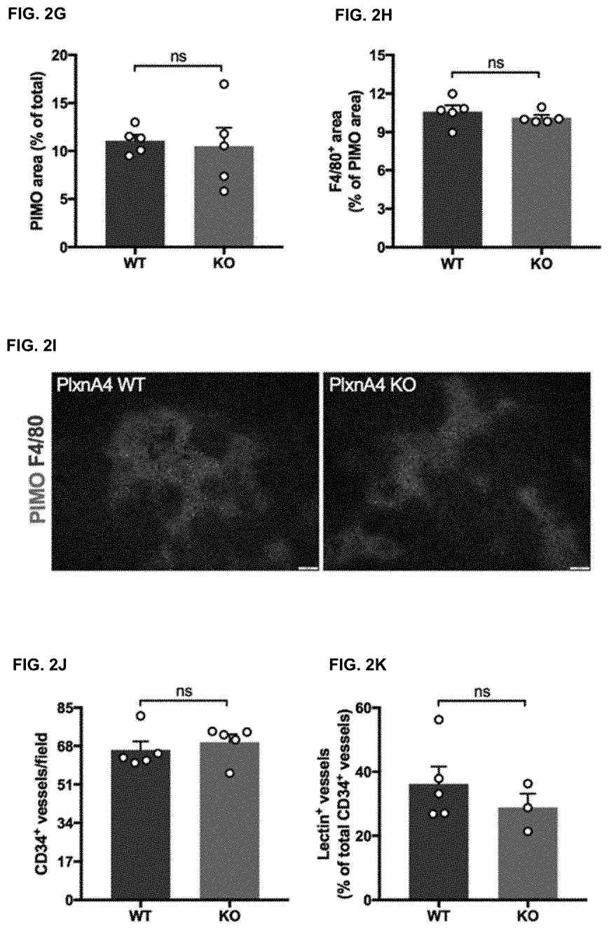

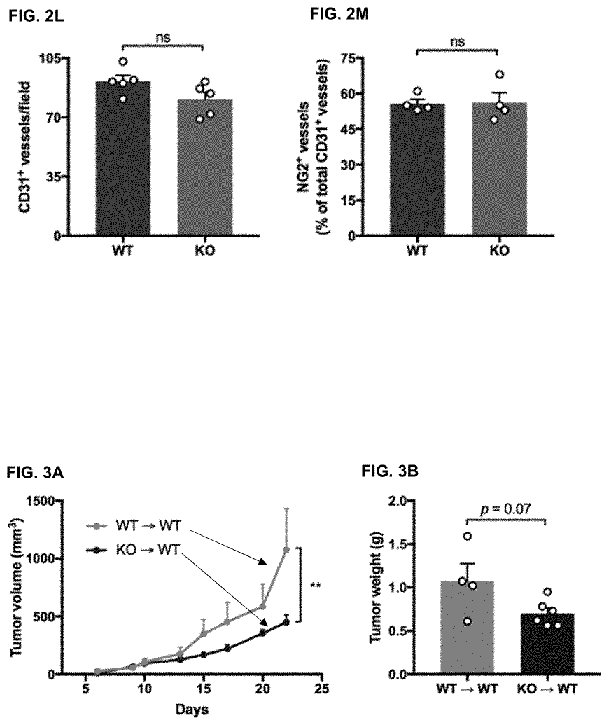

Because PlxnA4 was previously reported as part of the signaling cascade involved in the positioning of TAMs in the tumor, the impact of gene deletion on tumor macrophage infiltration (FIG. 2E), TAM phenotype (FIG. 2F), and localization within tumor niches (FIGS. 2H-21) was assessed. Staining of tumor sections from WT and Plxna4 KO mice for the macrophage-specific marker F4/80 showed neither difference in TAM infiltration of the primary tumor (FIG. 2E), nor in their localization within hypoxic regions (FIGS. 2H-21). Additionally, gene expression markers typically used to characterize classically (M1-like) and alternatively activated (M2-like) macrophages, were unaltered in sorted TAMs from WT and Plxna4 KO mice, suggesting that deletion of PlxnA4 does not affect macrophage polarization (FIG. 2F).

Previous observations suggest that PlxnA4 in human umbilical vein endothelial cells (HUVECs) may play a crucial role in bFGF-induced angiogenic sprouting of blood vessels (Kigel et al. 2011, Blood 118:4285-4296; WO2012114339A1). Hypoxic regions and blood vessel parameters were analyzed in WT and Plxna4 KO tumor-bearing mice. Tumor vessels were comparable between WT and Plxna4 KO mice with similar density (FIGS. 2J and 2L), vessel perfusion (FIG. 2K) and pericyte coverage (FIG. 2M), resulting in no differences in hypoxic areas (FIG. 2G).

In conclusion, loss of Plxna4 in the stroma reduces the tumor growth rate without affecting macrophage tumor infiltration, TAM phenotype, tumor vasculature nor the hypoxic areas.

TABLE 2
Hematological parameters in WT and Plxna4 KO mice.

| Cell Type | WT | Plxna4 KO | p-value |
|---|---|---|---|
| WBC (k/μL) | 3.63 ± 0.24 | 3.44 ± 0.25 | 0.59 |
| Neu (k/μL) | 0.21 ± 0.02 | 0.19 ± 0.02 | 0.64 |
| Lym (k/μL) | 3.23 ± 0.23 | 3.03 ± 0.24 | 0.54 |
| Mon (×10²/μL) | 0.67 ± 0.10 | 0.75 ± 0.13 | 0.65 |
| Eos (×10²/μL) | 0.81 ± 0.07 | 0.98 ± 0.10 | 0.17 |
| Bas (×10¹/μL) | 0.38 ± 0.13 | 0.15 ± 0.08 | 0.10 |
| RBC (M/μL) | 4.43 ± 0.05 | 4.46 ± 0.05 | 0.72 |

Data correspond to 20 mice per condition. Abbreviations: WBC, white blood cells; Neu, neutrophils; Lym, lymphocytes; Mon, monocytes; Eos, eosinophils; Bas, basophils and RBC, red blood cells. Data show mean ± SEM.

### Example 4. Deletion of PlxnA4 Exclusively in the Immune System Reduces Orthotopic Tumor Growth and Increases Infiltration of CD8+ T-Cell in Tumors

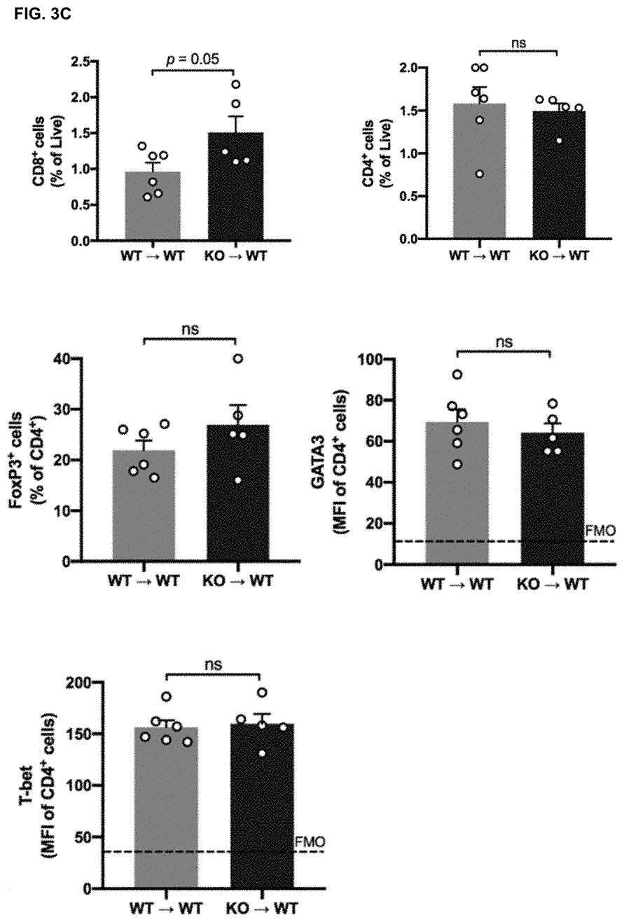

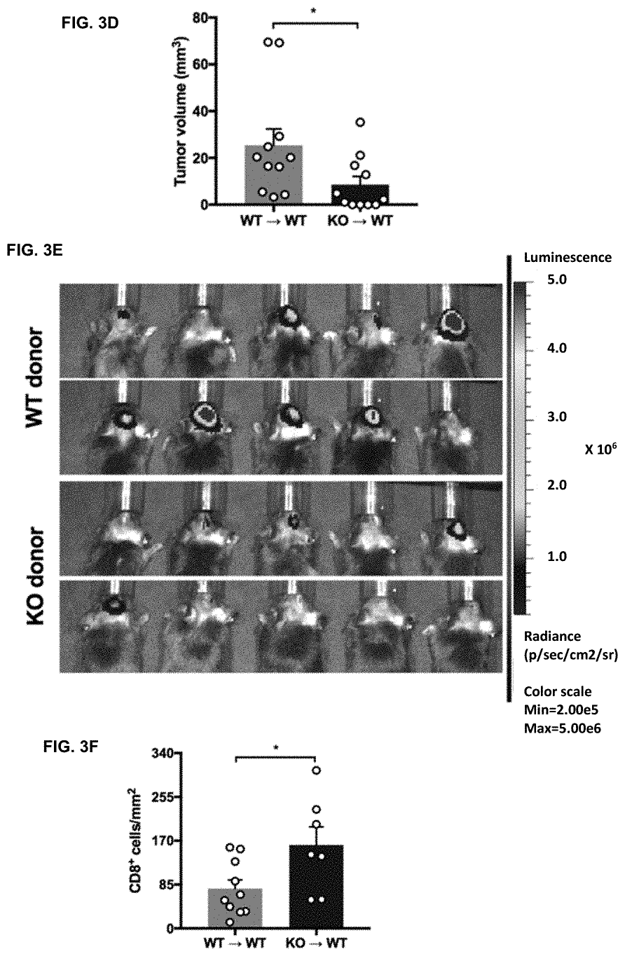

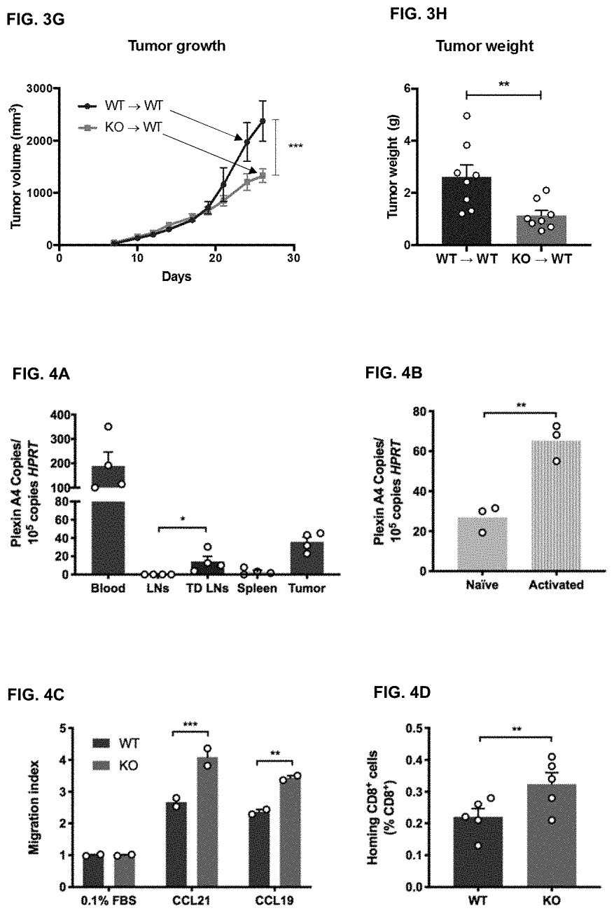

As a next step to decipher the role of PlxnA4 in the tumor stroma, a chimeric model was applied where PlxnA4 deletion is restricted to the immune system. Hereto bone marrow (BM) cells from WT or Plxna4 KO mice were transplanted into lethally irradiated recipient C57BL/6J mice, producing WT→WT or Plxna4 KO→WT mice, respectively. Upon reconstitution, Plxna4 KO→WT chimeras displayed normal blood counts and comparable to those of WT→WT mice (Table 3). As tumor microenvironment is reported to strongly influence tumor responses (e.g. Takahashi et al. 2018, Oncogene 37:2757-2772), it was evaluated how PlxnA4 loss in bone marrow-derived cells (BMDCs) affects the progression of orthotopic tumors. E0771 breast cancer cells were injected in the mammary fat pad of WT→WT or Plxna4 KO→WT mice. Consistent with the results observed for the Plxna4 KO mice, Plxna4 KO→WT mice showed reduced tumor growth comparing to their WT→WT counterparts (FIGS. 3A-3B). The immune infiltrate of E0771 tumors with the same tumor volume and weight was analyzed by flow cytometry. Tumors grown in Plxna4 KO→WT mice had increased infiltration of CD8⁺ cytotoxic T lymphocytes (CD8+ T-cells or CTLs,), with no differences observed in CD4+ T helper cells, or specific CD4+ T cell subsets, including regulatory T cells (Tregs) (FIG. 3C).

Because semaphorins are PlxnA4 ligands highly expressed in the brain, the effect of Plxn4 loss in an orthotopic immunological cold GL261 brain tumor model was analyzed. GL261 glioma cells were injected in the right striatum of the brain in WT→WT and Plxna4 KO→WT mice. Similar to what was observed for the orthotopic breast cancer model, tumors in the PIxnA4 KO chimeras grew significantly less comparing to the WT chimeras (FIGS. 3D-3E). Tumor sections where analyzed for CD8+ T cell infiltration, showing that tumors from Plxna4 KO→WT mice presented higher numbers of CD8+ T-cells comparing to the tumors grown in WT→WT chimeras (FIG. 3F).

Finally, in a subcutaneous LLC lung cancer model the loss of PlxnA4 in bone marrow-derived cells showed reduced tumor growth significantly different from their WT→WT counterparts (FIGS. 3G-3H). Together, these data show that deletion of PlxnA4 in the immune system increases CD8+ T-cell infiltration in tumors and is accompanied by a slower progression of the tumor growth independently of the tissue origin.

TABLE 3
Hematological parameters in WT→WT or Plxna4 KO→WT mice.

| Cell Type | WT → WT | Plxna4 KO → WT | p-value |
|---|---|---|---|
| WBC (k/μL) | 9.42 ± 0.65 | 9.65 ± 0.84 | 0.83 |
| Neu ((k/μL) | 0.40 ± 0.03 | 0.41 ± 0.06 | 0.87 |
| Lym ((k/μL) | 8.48 ± 0.60 | 8.73 ± 0.75 | 0.80 |
| Mon ((k/μL) | 0.11 ± 0.01 | 0.11 ± 0.02 | 0.98 |
| Eos (×10²/μL) | 0.18 ± 0.02 | 0.22 ± 0.04 | 0.44 |
| Bas ((k/μL) | 0.39 ± 0.03 | 0.32 ± 0.04 | 0.23 |
| RBC (M/μL) | 8.21 ± 0.74 | 9.23 ± 0.15 | 0.20 |

Data corresponds to 12 mice per condition. Abbreviations: WBC, white blood cells; Neu, neutrophils; Lym, lymphocytes; Mon, monocytes; Eos, eosinophils; Bas, basophils and RBC, red blood cells. Data show mean ± SEM.

### Example 5. PlxnA4 Deletion in CD8+ T-Cells Increases their Migratory and Proliferative Capacities Leading to a Stronger Anti-Tumor Response

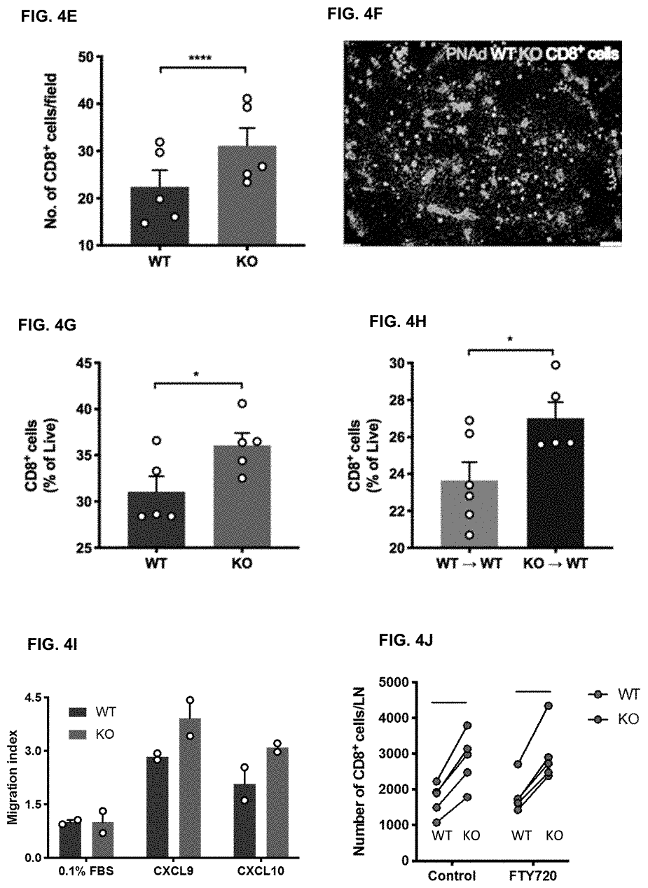

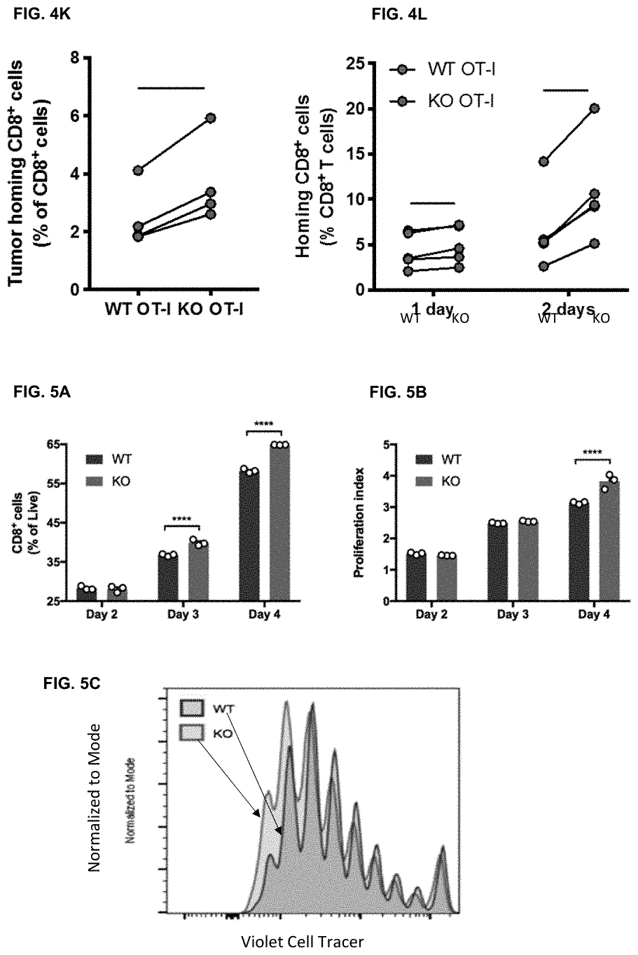

The expression of Plxna4 in CD8+ T-cells sorted from different organs from healthy and tumor-bearing mice was analyzed. In FIG. 4A is shown that Plxna4 is expressed in circulating CD8+ T-cells both in healthy and tumor-bearing mice, while its expression is significantly higher in the context of a tumor. No Plxna4 expression was detected in CD8+ T-cells from lymph nodes (LNs) and spleen in healthy mice (data not shown). In the context of a tumor, PlxnA4 expression was up-regulated in blood, tumor-draining LNs and in the TME (FIG. 4A). In vitro T-cell activation experiments showed a robust induction of Plxna4 expression in CD8+ T-cells after 4 days stimulation with CD3/CD28 (FIG. 4B). Together these data suggest an involvement of PlxnA4 in CD8+ T-cells activation in the LNs upon or during antigen presentation.

Since CD8+ T-cells expressed considerable levels of Plxna4 in the blood of tumor-bearing and healthy mice, a potential role in T cell motility was investigated in ex vivo chemotaxis assays using transwell plates. Migration of wildtype and PlxnA4 knockout CD8+ T-cells was assessed towards CCL21 and CCL19, chemokines involved in T cell homing to the LNs (Girard et al. 2012, Nat Rev Immunol 12:762-773), showing increased migration capacity of Plxna4-deficient CD8+ T-cells comparing to their WT counterparts (FIG. 4C). Moreover, Plxna4 KO CD8+ T-cells were more efficient in reaching the LNs upon transfer of WT and Plxna4 KO CD8+ T-cells into WT mice, as measured by entry into the LNs by flow cytometry and immunohistochemistry (FIGS. 4D-4E, and 4J).

In terms of localization, both WT and Plxna4 KO CD8+ T-cells were able to enter the paracortical areas of the LNs with no entrapment in the high endothelial venules (HEVs, FIG. 4F).

To analyse the migration capacity in tumor models, the total number of CD8+ T-cells in the LNs of mice bearing LLC subcutaneous tumors or E0771 orthotopic breast tumors was analyzed by flow cytometry of the tumor-draining LNs. Both Plxna4 KO mice and Plxna4 KO→WT chimeras showed increased numbers of CD8+ T cells in the draining LNs comparing to WT mice and WT→WT chimeras, respectively (FIGS. 4G-4H).

The migration capacity of PIexA4 KO CD8+ T-cells activated with CD3/CD28 was improved compared to activated WT CLTs in ex vivo chemotaxis assays towards CXCL9 and CXCL10, chemokines implicated in T cell recruitment to the TME (FIG. 4I).

In conclusion, PlxnA4 appears a negative regulator of CD8+ T-cell migration as loss of PlxnA4 in CD8+ T-cells was found to increase their migratory capacity towards the LNs, both in healthy and in tumor conditions.

Furthermore, the homing ability to the tumor of Plxna4 KO CD8+ T cells also showed to be increased in vivo, comparing to WT CD8+ T cells, in a competition assay in mice bearing lung (FIG. 4K) and melanoma tumors (FIG. 4L).

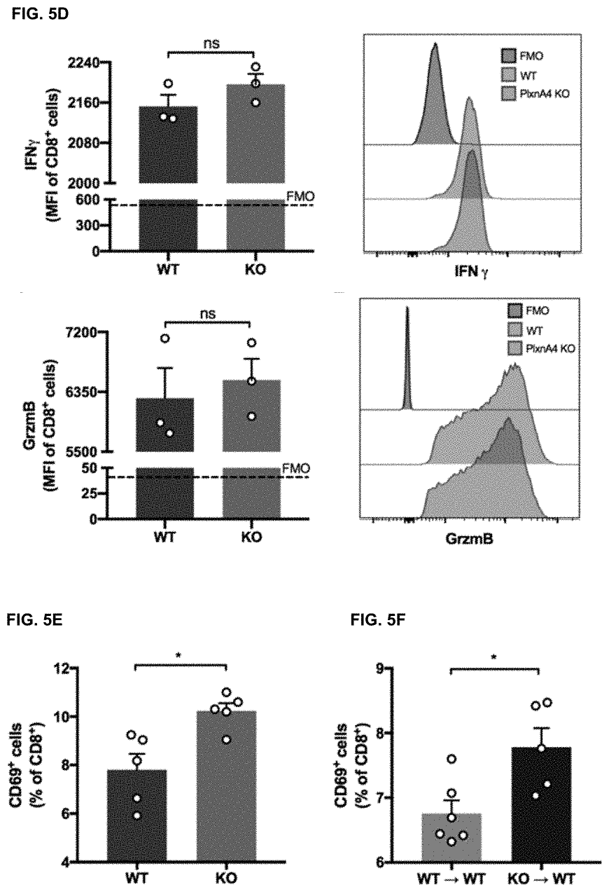

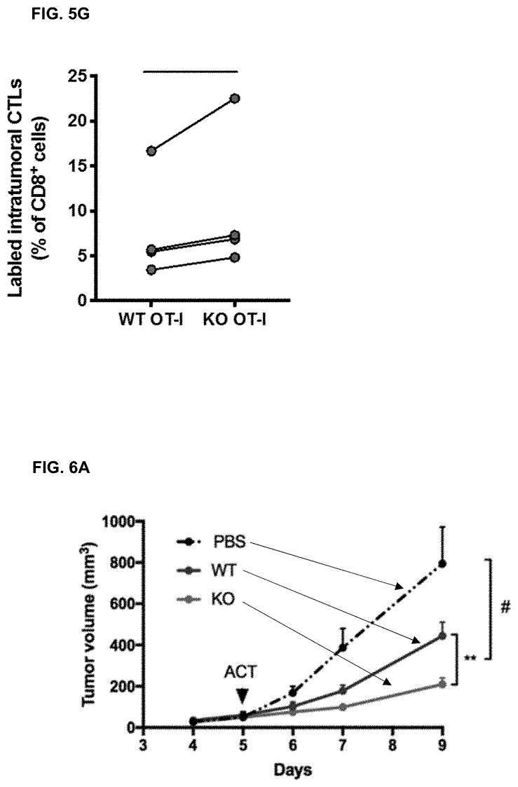

To assess whether the increased number of Plxna4 KO CD8+ T-cells in the tumors and in the LNs of tumor-bearing mice was a consequence of the increased migratory capacity of these cells, the in vitro proliferation of WT and Plxna4 KO splenocytes was analyzed in a time-course experiment. In the presence of primary and co-stimulatory signals (CD3/CD28 activation, respectively) optimized for efficient T-cell activation and expansion, the percentage of CD8+ T cells in the total splenocytes increased over time for both WT and Plxna4 KO cultures, with. Plxna4 KO CD8+ T-cells showing increased enrichments as of day 3 (FIG. 5A). The proliferation index showed increased proliferation of Plxna4 KO CD8+ T-cells compared to WT controls at day 4 upon activation (FIGS. 5B-5C). This time-point correlates with the increased expression of Plxna4 in CD8+ T-cells upon activation (FIG. 4B), which may suggest a negative regulation of this protein in CD8+ T-cell proliferation.

In vivo, the expression of the classical early marker of T-cell activation, CD69, showed to be upregulated in CD8+ T-cells in the LNs of KO tumor-bearing mice, compared to their respective WT controls (FIG. 5E). Furthermore, intratumoral injections of activated WT and Plxna4 KO CD8+ T cells, revealed that Plxna4 KO CTLs proliferate significantly more in the tumor bed as compared to WT ones (FIG. 5G). PlxnA4 has a cytoplasmatic region that contains a GTPase activating protein (GAP) domain, which mediates major intracellular signaling through the interaction with small GTPases (Kong et al. 2016, Neuron 91:548-560), and Rac1, a member of the small GTPases family, is necessary for the correct homing of the T cells to the LNs (Faroudi et al. 2010, Blood 116:5536-5547). The regulatory effect of PlxnA4 on the activation of small GTPases in CTLs was checked herein. For that, a GTPase pull down assay was performed to detect GTP-bound Rac1 in both WT and Plxna4 KO CTLs. Plxna4-deficient CD8⁺ T cells had increased levels of active Rac1 (GTP-bound) when compared with the WT ones. Of note, the levels of GTP-bound Rap1 were indistinguishable between both conditions. Altogether, these data further support the idea that PlxnA4 has a role in controlling CTL motility, via the downstream activity of Rac1 small GTPase.

Taken together, these results show that deletion of PlxnA4 in CD8+ T-cells increases their migratory capacity and induces a hyperproliferative response to TCR activation, without affecting their cytotoxicity in vitro. PlxnA4 KO CD8+ T-cells showed an increased activation status in the presence of an antigen, which is the case of draining LNs in tumor-bearing mice, suggesting that PlxnA4 is a negative regulator of CD8+ T-cells in the cancer context.

### Example 6. PIxnA4-Deficient CD8+ T-Cells have Enhanced Anti-Tumor Efficacy

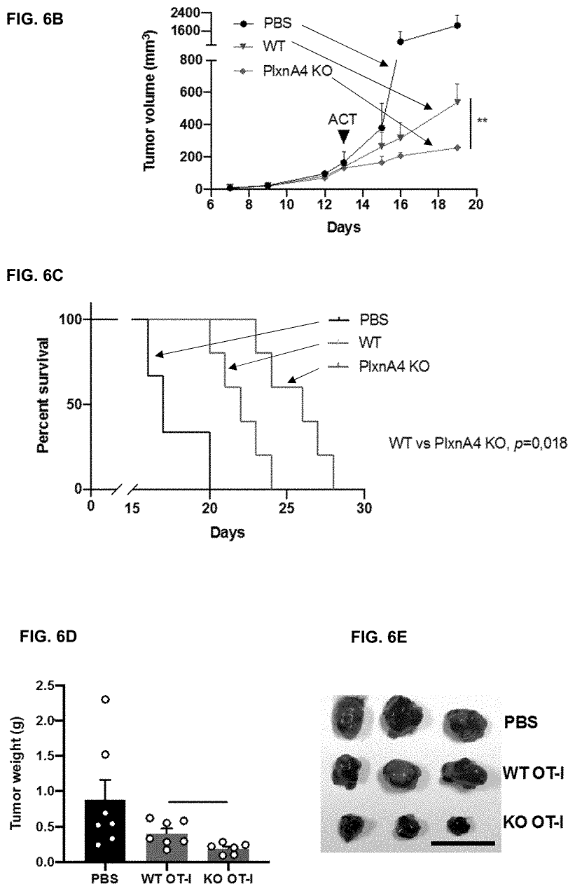

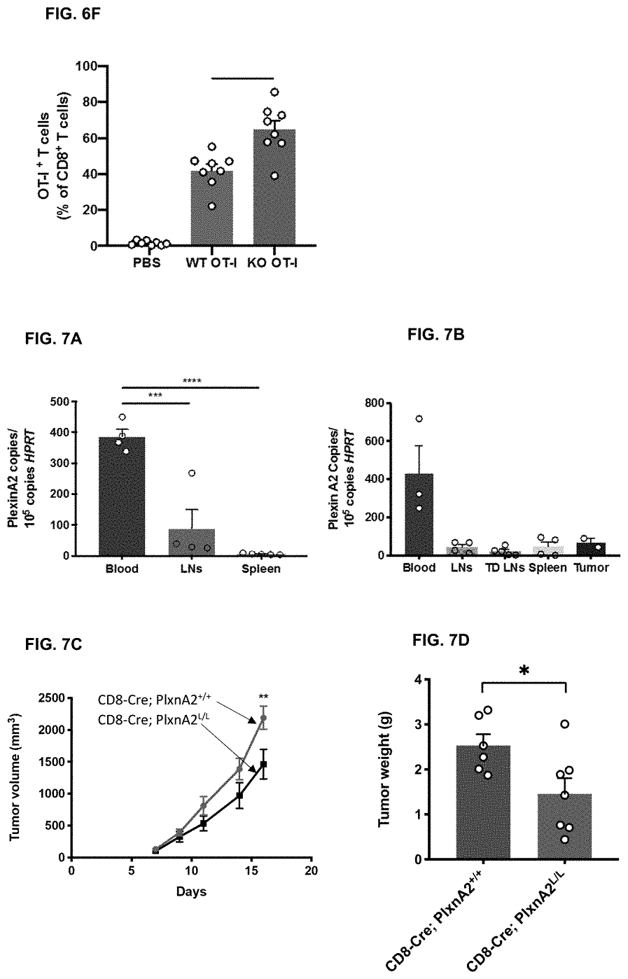

To evaluate the effect of deletion of PlxnA4 in CD8+ T-cells on the anti-tumor efficacy, the ability of Plxna4 deficient OT-1 CD8+ T-cells to control the tumor growth of LLC-OVA tumors was verified in an adoptive transfer regimen. Naïve Plxna4 KO OT-I CD8+ T-cells (CD8-positive T-cells expressing OT-1 and deficient in Plxna4) and the respective WT controls were transferred into LLC-OVA tumor-bearing WT recipient mice to monitor tumor progression. The transfer of Plxna4 KO OT-I CD8+ T-cells lead to a strong abrogation of the normal tumor growth, in comparison to the PBS group (FIG. 6A). Wild-type OT-I cells were also able to control tumor growth, but to a significantly lesser extent than the Plxna4 KO OT-I CD8+ T-cells (FIG. 6A). This shows the increased capacity of PlxnA4 KO CD8+ T-cells to migrate towards the LNs and reach the tumor.

In a more therapeutic approach, activated PlxnA4 WT and KO OT-I T cells were adoptively transferred in B16-F10 melanoma tumor-bearing mice. In this setting, adoptive transfer of Plxna4 KO OT-I CD8+ T-cells likewise was able to control tumor growth to a significantly higher extent than wild-type OT-I CD8+ T-cells, resulting in an increased overall survival of the mice (FIGS. 6B-6E), and an increased number of intratumoral KO OT-I CD8+ T-cells (compared to the number of wild-type OT-I CD8+ T-cells) (FIG. 6F). Together these data show that the selective deletion of PlxnA4 in CD8+ T-cells is sufficient to increase anti-tumor immunity in two distinct tumor models, and that targeting PlxnA4 in CD8+ T-cells appears as a valuable strategy to manage several tumor types, including immunologically cold tumors.

### Example 7. Conditional PlxnA2 Deletion in CD8+ T-Cells Leads to Enhanced Infiltration of CD8+ T-Cells in Tumors and to Reduced Tumor Growth

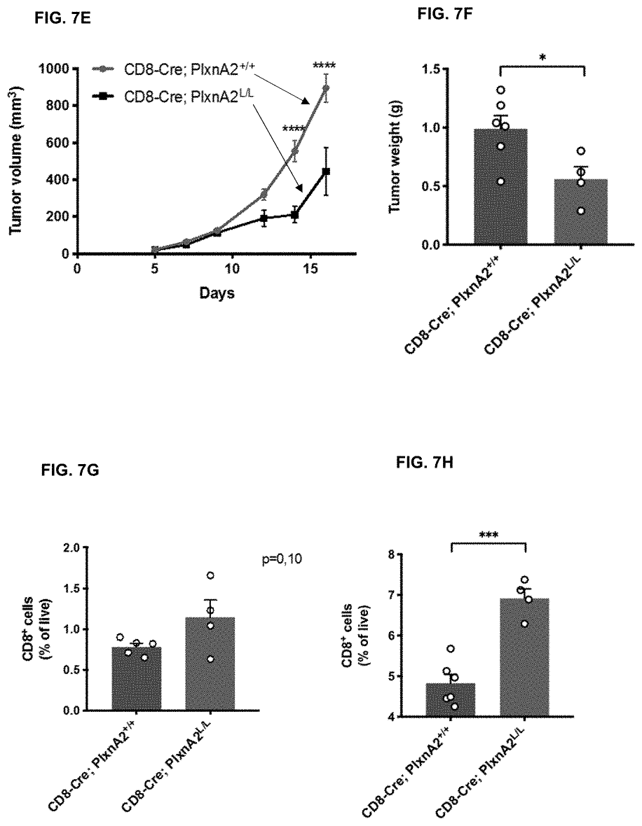

PlexinA2 shares the same ligands and signalling cascade as PlexinA4, and has been reported to be able to form heterodimers with PlexinA4. To assess a potential role of PlexinA2 on CD8+ T-cells in the tumor microenvironment, mRNA expression of plexinA2 was analysed after flow cytometric cell sorting of CD8⁺ Tcells derived from different tissues from either LLC-tumor bearing mice and healthy mice. Results shown in FIGS. 7A and 7B indicate that PlxnA2 is highly expressed in circulating CD8+ T cells in healthy and tumor-bearing mice.

To directly decipher the functionality of Plxn2 expressed on CD8+ T cells in a cancer setting, a conditional knockout model was set-up using the Cre-lox system, PlexinA2 LA CD8.Cre KO mouse model. Tumor growth was monitored in two distinct syngeneic tumor models, subcutaneous MC38 colon adenocarcinoma (FIG. 7C, 7D) and orthotopic E0771 TNBC (FIG. 7E, 7F). In both models the PlxnA2-specific deletion in CD8+ cells was found to reduce the tumor growth versus the wildtype control group. The analysis of tumor-infiltrating CD8+ T-cells was done by flow cytometry in orthotopic E0771 tumors grown until day 16. FIG. 7G-7H shows the results. The PlxnA2-specific deletion in CD8+ T cells leads to a higher number of CD8+ T cells in blood and primary tumors compared to the WT controls.

In conclusion, the selective loss of PlxnA2 on CD8+ T-cells enhances their tumor infiltration and reduces the tumor growth.

### Example 8. Bispecific PlxnA4-CD8 Antibodies

### 8.1. Generation of Bispecific PlxnA4-CD8 VHHs

In order to therapeutically inhibit the PlexinA4 function on immune cells, we generated a panel of PlexinA4-specific VHHs.

To this end, PlexinA4-specific VHHs were isolated from the immune repertoire of llama's that had been immunized with a recombinant human PlexinA4 extracellular domain (ECD) with a C-terminal His6 tag (Cat. Nr. 5856-PA-050, R&D systems) and/or mouse PlexinA4 extracellular domain with a C-terminal His₆ tag (generated in-house, aa 24-1233, Q80UG2.3) using the phage display technology.

Following two selection rounds of immune libraries on biotinylated antigens, screening of individual clones was done in binding ELISA to identify specific binders to human and/or mouse PlexinA4 ECD.

To direct the functional blockade of PlexinA4 towards cytotoxic T cells (CTLs), bispecific VHH constructs were generated in which a PlexinA4-specific VHH was genetically fused to a CD8-binding VHH that is not interfering with CD8 function. Six distinct PlexinA4-specific VHHs (PLX1 to PLX6 (see Table 4) with less than 90% overall amino acid sequence identity and substantial differences across the three CDRs) were selected for formatting into bispecific formats with a single human CD8 alpha chain-specific VHH (WO 2019/032661, clone 3CDA5). As reference constructs, each of the PlexinA4 VHHs was also formatted with an irrelevant control VHH. Two PlexinA4 VHHs were in addition formatted with a human CD4-specific VHH (WO 2015/044386, clone 3F11), to be able to confirm specificity towards CD8+ T cells. In each case, the two VHH moieties were genetically linked with a single flexible glycine-serine linker ([glycine₄-serined₄, referred to as 20GS linker), with a C-terminal Flag₃-His₆ tag. The panel of bispecific VHH constructs is listed in Table 4.

Bispecific VHH constructs were introduced in the cDNA3.4 vector for expression in 293F cells, and culture supernatants were purified by HisTrap fast flow affinity chromatography, followed by desalting. Monovalent VHHs were produced in E. coliTG-1 strain at 200 mL scale, and VHHs were purified from the periplasmatic extracts by immobilized metal affinity chromatography on Nickel-sepharose (Robocolumn, Repligen), followed by desalting. Protein integrity and purity was confirmed by SDS-PAGE under non-reducing conditions, and quantification was done using Bradford method and Nanodrop. Western blot analysis confirmed the integrity of the flag and His-tags.

TABLE 4
Overview of bispecific PlexinA4-CD8 VHH
constructs, and controls

| | | monovalent |
|---|---|---|
| bispecific | | plexinA4-specific |
| VHH # | bispecific VHH construct | VHH unit |
| 1 | PLX1-20GS-CD4-FLAG-HIS6 | PLX1 |
| 2 | PLX1-20GS-CD8-FLAG-HIS6 | |
| 3 | PLX1-20GS-IRR-FLAG-HIS6 | |
| 4 | CD8-20GS-PLX1-FLAG-HIS6 | |
| 5 | PLX2-20GS-CD4-FLAG-HIS6 | PLX2 |
| 6 | PLX2-20GS-CD8-FLAG-HIS6 | |
| 7 | PLX2-20GS-IRR-FLAG-HIS6 | |
| 8 | PLX3-20GS-CD8-FLAG-HIS6 | PLX3 |
| 9 | PLX3-20GS-IRR-FLAG-HIS6 | |
| 10 | PLX4-20GS-CD8-FLAG-HIS6 | PLX4 |
| 11 | PLX4-20GS-IRR-FLAG-HIS6 | |
| 12 | PLX5-20GS-CD8-FLAG-HIS6 | PLX5 |
| 13 | PLX5-20GS-IRR-FLAG-HIS6 | |
| 14 | PLX6-20GS-CD8-FLAG-HIS6 | PLX6 |
| 15 | PLX6-20GS-IRR-FLAG-HIS6 | |
| 16 | IRR-20GS-CD4-FLAG-HIS6 | none |
| 17 | IRR-20GS-CD8-FLAG-HIS6 | none |

### 8.2. Binding of Bispecific PIxnA4-CD8 Towards PlexinA4 in Biolayer Interferometry

The binding of the panel of purified bispecific VHHs towards recombinant human PlexinA4 ECD was assessed via biolayer interferometry on an Octet RED96 system (FortéBio). Anti-streptavidin capture biosensors (Cat nr. 18-0009, FortéBio) were soaked in kinetics buffer (10 mM HEPES pH 7.5, 150 mM NaCl, 1 mg/ml bovine serum albumin, 0.05% Tween-20 and 3 mM EDTA) for 20 min. Biotinylated human PlexinA4 ECD (Cat. Nr. 5856-PA-050, R&D systems) at 5 μg/ml was immobilized on these AMC biosensors to a signal of 1.5 nm. Association (400 s) and dissociation (1200 s) of 100 nM of each bispecific construct in kinetics buffer were measured at 30° C. For comparison, the corresponding purified monovalent anti-PlexinA4 VHHs were assessed at 100 nM alongside the bispecific constructs. Data were double reference-subtracted and aligned to each other in Octet Data Analysis software v9.0 (FortéBio) based on a baseline measurement of blanc control. Association and dissociation of non-saturated curves were fit in a global 1:1 model.

The results of kinetic binding profile determination and of dissociation constants determination of the bispecific VHH constructs in comparison to the monovalent plexinA4 VHH indicated that binding towards human PlexinA4 is preserved in the bispecific formats, with off-rates ranging between 2.2×10⁻³ 1/s and <1×10⁻⁶ 1/s, essentially following the monovalent PlexinA4 VHHs.

8.3. Binding of Bispecific PlxnA4-CD8 towards PlexinA4 Expressed on Cells

To assess the binding of bispecific VHH polypeptides to cell-expressed human PlexinA4, HEK293T cells were transiently transfected with a plasmid encoding full length human PlexinA4.

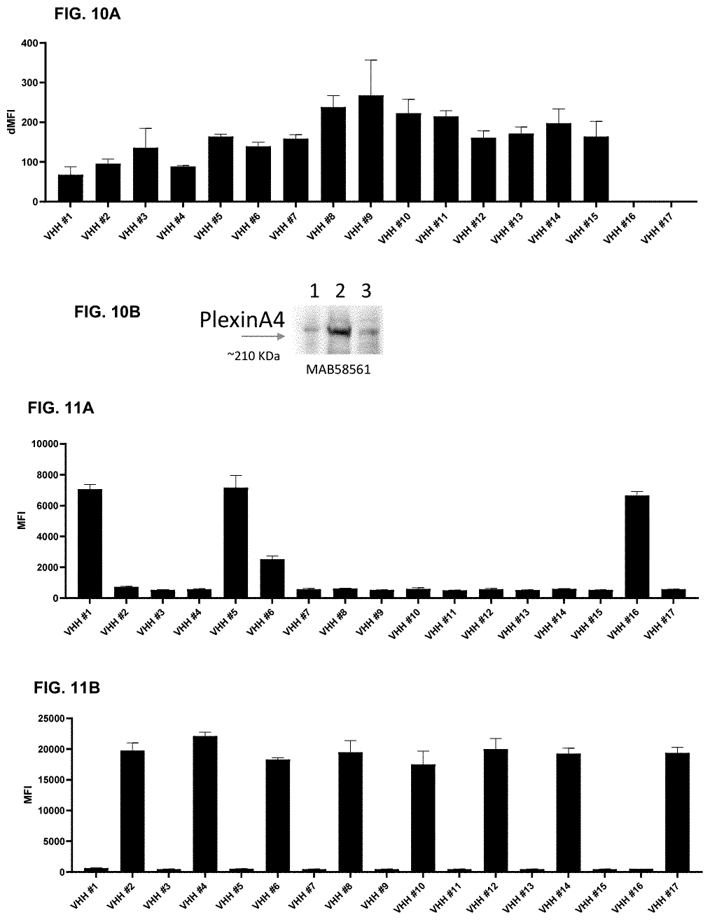

To this end, HEK293 cells were transfected with a human PlexinA4 (Q9HCM2.4) expressing vector (Invitrogen #20AA5QSP) or empty vector using lipofectamine (Invitrogen). Expression of the human PlexinA4 protein on the transfected cells was confirmed by western-blot analysis on the whole cell lysates using the anti-hPlexinA4 monoclonal antibody (R&D Systems MAB58561) for detection (FIG. 10B).

For the cell binding analysis of the different bispecific VHH constructs, a three-step binding flow cytometry protocol was performed. First, hPlexinA4-HEK293 cells and mock control cells were incubated with 200 nM single dose of the different bispecific VHH molecules for 30 minutes at 4° C. in FACS buffer (PBS, 10% FBS). Next, cells were washed by centrifugation and probed with anti-FLAG antibodies (BioM2 biotin conjugated, Cat. nr. F9291 Sigma-Aldrich) for 30 minutes at 4° C., to detect bound VHH. Detection was done with Streptavidin-AF488 (Invitrogen, S32354) for 30 minutes at 4° C. Cells were washed and incubated with TOPRO3 to stain for dead cells, which are then removed during the gating procedure. Cell binding was analyzed via a FACS Celesta (BD), and the median fluorescence intensity (MFI) was determined. Results were normalized by subtraction of the background MFI of mock-transfected HEK293 cells with empty vector (dMFI). The results of hPlexinA4-HEK293 cell binding by the different VHHs are depicted in FIG. 10A. PlexinA4 target binding is confirmed for the different bispecific VHH constructs comprising a PlexinA4-binding VHH module, but not for the control molecules which lack a plexinA4-specific VHH, and did not show increased signal compared to the pCDNA3 (empty control vector) transfected cells.

### 8.4. Binding of Bispecific PIxnA4-CD8 Towards CD8 Expressed on Human T Cells

To assess the binding of bispecific VHH constructs to cell-expressed human CD8, binding of the bispecific VHH's to CD4+ and CD8+ specific T cell subsets was determined by flow cytometry on human activated primary T cells.

Human primary peripheral blood mononuclear cells (PBMCs Lonza, #4W-270) were activated with anti-human CD3 (HIT3a, Biolegend) and anti-CD28 (CD28.2, Biolegend) during 90 hours. A single dose of 10 μg/ml of the different bispecific VHH was added to the media on days 1 and 3 during activation. Detection of the binding by FACS analysis was performed on day 4. Briefly, cells were incubated with Fc-blocking solution for 15 minutes (Miltenyi #130-059-901) before staining for T cell markers surface expression. The staining included, anti-CD3 PerCP-Cγ5.5 (Biolegend #300328), anti-CD8 PE-Cγ7 (Biolegend #100722), anti-CD4 APC (Biolegend, #357408) in combination with anti-FLAG (BioM2 antibody biotin conjugated, Sigma-Aldrich) and NIR-zombie (Biolegend #423105) for dead cell exclusion for 30 minutes at 4° C. Cells were washed and incubated with StrepAF488 (Invitrogen, S32354) for the detection of VHH binding for 30 minutes at 4° C. Samples were analyzed with a BD FACSCelesta. Binding was determined by the MFI (median fluorescence intensity) on the different T cell populations. In the results shown in FIG. 11, the specific binding to either the CD4+ T cell subset (FIG. 11A) or CD8+ T cell subset (FIG. 11B) is confirmed for the different PlexinA4-bispecific VHH. The anti-hCD8 antibody clone RPA-T8 could not be used to detect CD8+ T cells, as this antibody competed with the hCD8 VHH for binding towards CD8. Bispecific VHH constructs comprising the CD4-VHH showed preferential binding in CD4+ T cells, while bispecific VHH molecules comprising the CD8-VHH showed superior binding levels in CD4-T cells, corresponding to CD8+ T cells. The constructs where PlexinA4 VHHs are fused to irrelevant VHH do not show detectable binding, indicating PlexinA4 is not (detectably) expressed on these cells.

8.5. Bispecific VHHs Compete with Ligand Binding to Human PlexinA4

To assess the capacity of the PlexinA4 VHHs and bispecific VHH constructs to inhibit binding of PlexinA4-ligands with PlexinA4, dose-dependent competition of semaphorin 6a-binding to PlexinA4 was assessed using an Alphascreen method (Perkin Elmer). Herein, biotinylated human PlexinA4 ECD (Cat No 5856-PA-050, R&D systems) at a final concentration of 10 nM was incubated with serial dilutions of monovalent or bispecific VHH constructs for 1 hour at room temperature. As positive control served recombinant human PlexinA4 Sema-PSI-1 domain (Q9HCM2; residues 24-559, in-house production). The mixture of PlexinA4 and VHHs was transferred to 384-well F-bottom white plates (Greiner, Cat no 781904), after which recombinant human Semaphorin 6A-Fc protein (R&D Systems, Cat no 1146-S6) was added to a final concentration of 3 nM. After mixing and incubation for 1 hour at room temperature, streptavidin coated alpha donor beads (Perkin Elmer, Cat no 6760002) and anti-human IgG (Fc specific) Alpha LISA acceptor beads (Perkin Elmer, 6760002) were added to a final concentration of 20 μg/mL each, following by an incubation for 1 hour at room temperature in the dark. Excitation was done at 680 nm, with emission signals were read-out at 611 nm on Ensight, according to the manufacturer's recommendations (Perkin Elmer).

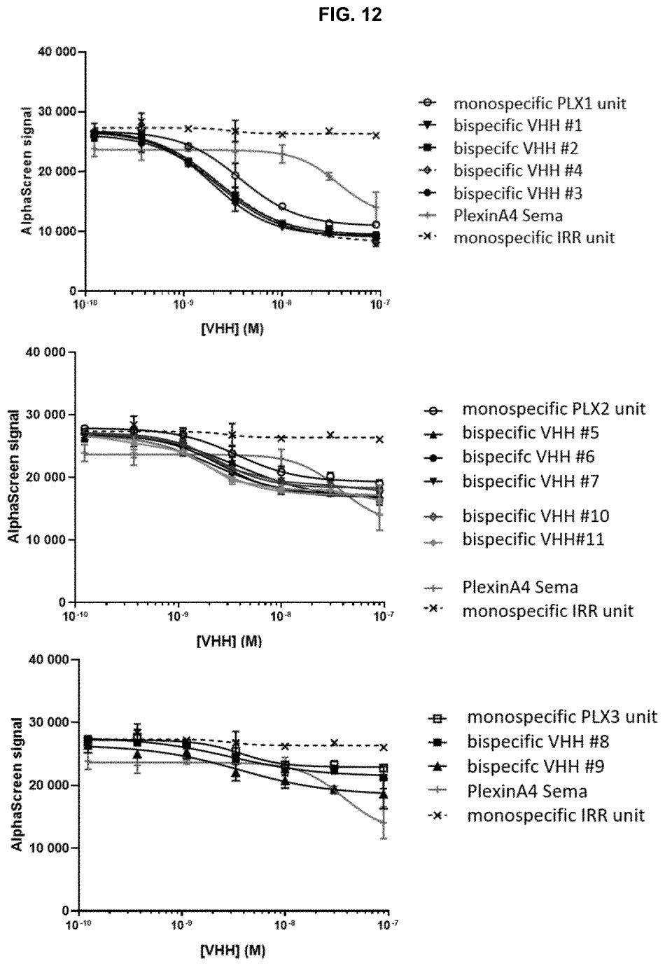

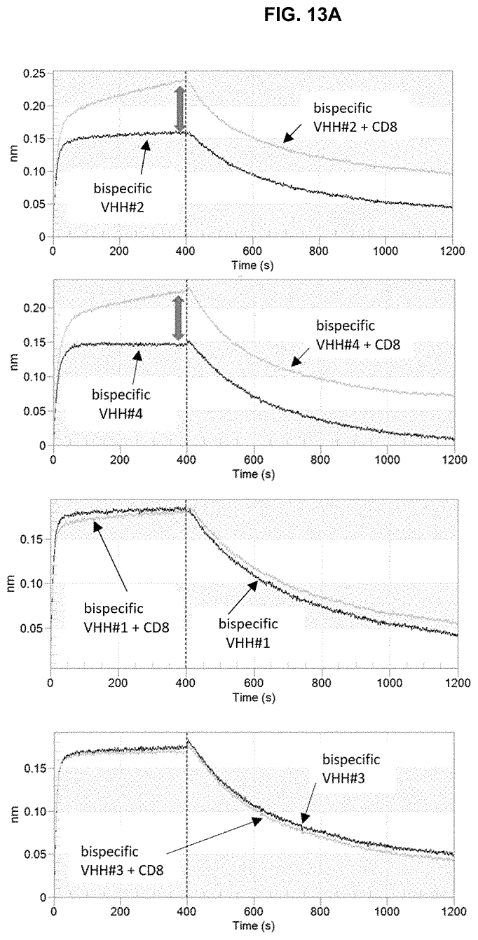

Results are depicted in FIG. 12. The ligand competition exerted by the bispecific VHH molecules essentially follows the competition exerted by the monovalent plexinA4 VHH units, with constructs comprising the PLX1-VHH unit showing the strongest Semaphorin6a competition (up to 70% inhibition efficacy), with IC₅₀ values ranging between 1.9-2.3 nM. The constructs comprising the anti-PlexinA4 VHH units PLX2, PLX4, and PLX3 are partial inhibitors of Semaphorin 6a interaction (inhibition efficacy between 20-40%), showing lower inhibition than the human PlexinA4 Sema-PSI-1 domain used as reference.

### 8.6. Bispecific VHHs can Simultaneously Bind to PlexinA4 and CD8 in Biolayer Interferometry

To assess the simultaneous binding of the purified bispecific VHHs towards both human PlexinA4 ECD and human CD8 protein, we used biolayer interferometry on an Octet RED96 system (FortéBio). Herein, biotinylated human PlexinA4 ECD (Cat No 5856-PA-050, R&D systems) at 5 μg/ml was immobilized to anti-streptavidin capture biosensors (Cat no 18-0009, Forte Bio) were soaked in kinetics buffer (10 mM HEPES pH 7.5, 150 mM NaCl, 1 mg/ml bovine serum albumin, 0.05% Tween-20 and 3 mM EDTA) for 20 min on these AMC biosensors to a signal of 1.5 nm. The association of bispecific VHH constructs at 100 nM alone or pre-mixed with 100 nM recombinant human CD8 alpha/beta protein (Cat no 9358-CD-050 R&D Systems) was assessed for 400 s. The simultaneous binding to both PlexinA4 ECD and CD8 proteins is reflected by a higher binding level in the association phase.

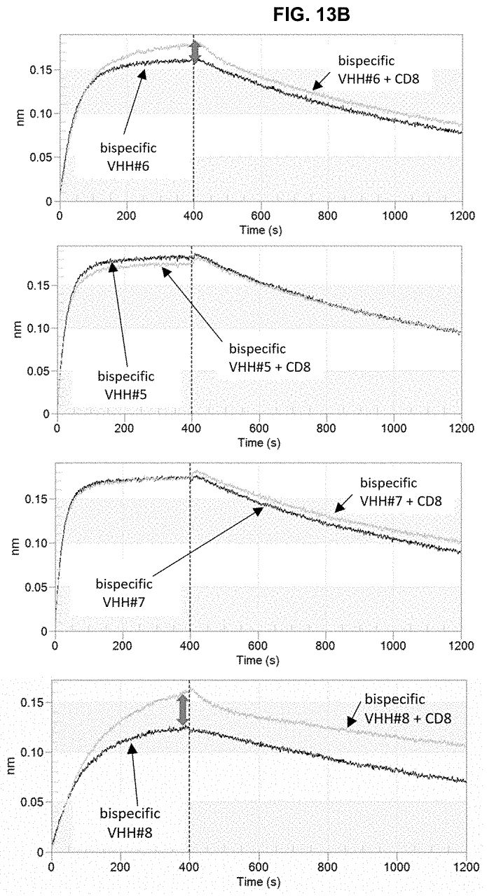

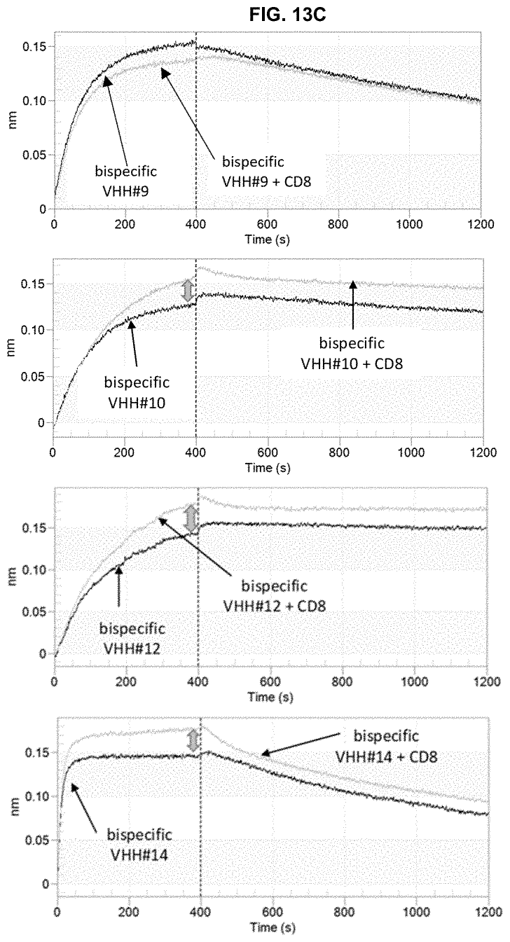

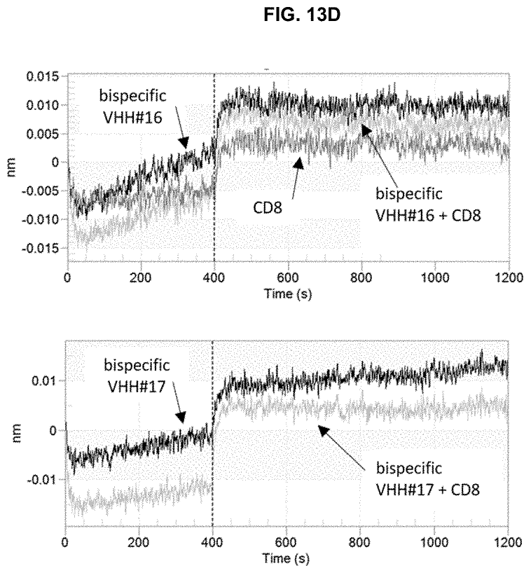

FIG. 13 depicts the results for the different bispecific VHH constructs (as listed in Table 4). The results indicate that bispecific VHH constructs combining PlexinA4 and CD8 VHHs can simultaneously bind to both proteins, while the constructs with the irrelevant control VHH or CD4 VHH cannot. Together with the results from Examples 8.2, 8.3 and 8.4, this confirms that Plexin-A4/CD8 bispecific antibodies were generated retaining the capacity to bind both to Plexin-A4 and CD8 on cells. Furthermore, as evident from Example 8.5, these Plexin-A4/CD8 bispecific antibodies are interfering with the binding of a Plexin-A4-ligand to Plexin-A4. As such, these Plexin-A4/CD8 bispecific antibodies are compounds inhibiting plexin-A4, wherein these compounds are specifically targeting plexin-A4 on CD8-positive (CD8+) T-cells.

### Example 9. Effect of Bispecific PlexinA4-CD8 VHH Constructs on the Migration Capacity of CD8+ T Cells

To verify that bispecific PlexinA4-CD8 VHHs show increased specificity for T-cells co-expressing both receptors, the effect of bispecific VHHs on the migratory capacity of human CD8+ T-cells is evaluated (as described in Example 5 hereinabove, or as described in Leclerc et al. 2019, Nat Commun 10:3345, or alike). To this end chemotaxis assays towards different chemokines implicated in T-cell recruitment to the tumor micro-environment (TME) (such as CXCL12, CXCL10, CXCL9) are analyzed. To this end, CD3/CD28 activated human T-cells from different donors are incubated with the different bispecific PlexinA4-CD8 molecules. Following incubation for 30 minutes to 1 hour, cells are transferred to the top chamber of transwell plates (Corning, CLS3421). Low serum medium (0.1% FBS) with different chemokines in the lower chamber is used to trigger T-cell migration. After 2 hours at 37° C. the migrated cells are collected for counting. As PlexinA4 ligands (Sema 3A, Sema 6A and Sema 6B) are upregulated in the TME, they are expected to affect the function of plexinA4 in T-cell migration towards chemokines within the TME. For this reason, the effect of the PlexinA4 blockade in the T-cell migratory capacity is assessed in the presence of the different semaphorin ligands in comparison to an isotype hIgG control. As T-cell subsets have been reported to produce endogeneously Semaphorin 3a in mixed leukocyte cultures, the Plexin A4 blockade is also assessed in the absence of exogenous ligands. Simultaneous binding of the bispecific PlexinA4-CD8 VHH to plexinA4 and CD8 increases migration toward chemokines. To discriminate which T-cell subset benefits from increased migratory capacity upon the different treatments, FACS analysis including T-cell markers is carried out on the migrated T-cell compartment.

1. A composition comprising a compound which specifically reduces plexin-A2 and/or plexin-A4 activity on or in CD8-positive (CD8+) T-cells.

2. The composition of claim 1,
wherein the compound is a polypeptide, a polypeptidic agent, or an aptamer binding to plexin-A2 and/or plexin-A4; and wherein the compound induces degradation of plexin-A2 and/or of plexin-A4; or
wherein the compound interferes with expression of plexin-A2 and/or of plexin-A4.

3. The composition of claim 1, wherein the compound is selected from the group consisting of a polypeptide comprising an immunoglobulin variable domain, an antibody or a fragment thereof, an alpha-body, a nanobody, an intrabody, an aptamer, a DARPin, an affibody, an affitin, an anticalin, a monobody, a bicyclic peptide, a PROTAC, a LYTAC; and a combination of any of the foregoing.

4. The composition of claim 1, wherein the compound further comprises a moiety that specifically binds to a CD8+ T-cell-specific surface marker other than plexin-A2 and/or plexin-A4.

5. The composition of claim 4, wherein the CD8+ T-cell-specific surface marker other than plexin-A2 and/or plexin-A4 is CD8 or CD69.

6. The composition of claim 1, wherein the compound specifically reduces plexin-A2 and/or plexin-A4 activity on or in CD8+ T-cells in a tumor and/or in the tumor micro-environment of a subject having a tumor.

7. A method a administering the composition of claim 1 to a subject, the method comprising:
administering the composition to a tumor and/or a tumor micro-environment in the subject by means of intra- or peri-tumoral administration; or
administering the composition to the subject wherein the composition further comprises a carrier, wherein the cargo of the carrier is the compound,
wherein the carrier targets its cargo to a tumor and/or a tumor micro-environment in the subject, and/or wherein release of the cargo from the carrier occurs in the tumor and/or in the tumor micro-environment.

8. The method according to claim 7, wherein the carrier is a virus, an oncolytic virus, cells adoptively transferred to the subject, exosomes, nanoparticles, or microbubbles.

9. The composition of claim 1, wherein the composition is a pharmaceutical composition.

10. The composition of claim 1, wherein the composition further comprises an anticancer agent.

11. (canceled)

12. The method according to claim 7, wherein the administration of the composition to the subject treats, inhibits, or suppresses the tumor or a cancer.

13. The method according to claim 7, further comprising treating the subject with surgery, radiation, chemotherapy, targeted therapy, immunotherapy, or an anticancer agent.

14. (canceled)
